(19) United States
(12) Patent Application Publication
(10) Pub. No.: US 2026/0258604 A1
(43) Pub. Date: Sep. 3, 2026

(54) CELLULOSE-CONTAINING COMPOSITIONS, METHODS FOR THEIR PREPARATION, AND USES COMPRISING THEM

(71) Applicant: RENMATIX, King of Prussia, PA (US)

(72) Inventors: Danilo Alberto Cantero, Arroyo de la Encomienda (ES); Mark Daniel Cannatelli, Dandenong (AU); Michael Lee Smith, Kennesaw, GA (US)

(21) Appl. No.: 18/881,600

(22) PCT Filed: Jul. 5, 2023
(86) PCT No.: PCT/US2023/026922

§ 371 Date: Jan. 6, 2025

Related U.S. Application Data
(60) Provisional application No. 63/358,558, filed on Jul. 6, 2022.

(51) Int. Cl. D21C 9/00; A23L 29/20; A61K 8/73; A61Q 19/00

(52) U.S. Cl. CPC D21C 9/007; A23L 29/20; A61K 8/731; A61Q 19/007; D21C 9/002

(57) ABSTRACT

The present invention cellulose-containing compositions, processes for their preparation from cellulose-containing feedstocks, as well as thickeners, suspensions, emulsions, food products, personal care and cosmetic products comprising them.

### CROSS-REFERENCE TO RELATED APPLICATIONS

This application claims the benefit of U.S. Application No. 63/358,558, filed on Jul. 6, 2022, the contents of which are incorporated herein by reference in their entirety.

### FIELD OF THE INVENTION

The present invention is related to cellulose-containing compositions, methods for their preparation from cellulose-containing feedstocks, and uses comprising them.

### BACKGROUND OF THE INVENTION

Cellulose-containing materials merit special consideration in the global concern over the environment and raw materials, because they are renewable, biodegradable, and are the world's most abundant natural polymer. It has been estimated that between about 10¹⁰ to about 10¹¹ tons of cellulose is consumed globally each year in industrial applications for making textiles, paper products, plastics, food and pharmaceutical additives, cosmetic additives, propellants, and as an affordable renewable energy source.

Lignocellulosic biomass typically contains cellulose, hemicellulose, lignin, and minerals, and in some instances minor amounts of proteins and lipids (fats, waxes, and oils). About two thirds of the dry mass of lignocellulosic materials is present as cellulose and hemicellulose with lignin making up the bulk of the remaining dry mass. There are a number of processes for converting lignocellulosic biomass into liquid streams of various sugars, extracting lignin, and/or recovering unreacted cellulose, such as in the pulp and paper industry. However, despite the widespread utility of lignocellulosic materials, the available conversion processes are complicated, capital intensive, time consuming, and typically require the use of harsh toxic chemicals. Therefore, there is a need for compositions containing cellulose, environmentally friendly methods for preparing them cleanly and efficiently, and applications for using them.

### SUMMARY OF THE INVENTION

The present invention provides cellulose-containing compositions, processes for their preparation from cellulose-containing feedstocks, as well as thickeners, suspensions, emulsions, food products, personal care and cosmetic products comprising them. The processes of the present invention produce cellulose-containing compositions having a small particle size distribution, and unexpectedly produce these particles at lower temperatures and shorter reaction times than previously available.

### DETAILED DESCRIPTION

An embodiment of the present invention provides a method for the preparation of a cellulose-containing composition comprising the steps of: (a) forming a first slurry comprising a cellulose-containing feedstock, water, and acid; (b) subjecting the first slurry to rapid heating to a first temperature, by addition of subcritical, near critical or supercritical water, at a first pressure thereby forming a second slurry; (c) maintaining the second slurry at the first temperature and first pressure for a first time period; (d) rapidly depressurizing the second slurry to form a third slurry at a second temperature and a second pressure; and (e) separating the liquid portion from the solid portion of the third slurry, the solid portion of which comprises the cellulose-containing composition; wherein the first temperature is in the range of 180° C. to 280° C.; and wherein the cellulose contained in the cellulose-containing composition has a weight average molecular weight between 40,000 grams/mole and 155,000 grams per mole.

In a further embodiment of the present invention, the cellulose-containing composition prepared by the method of the present invention has a weight average molecular weight of between 75,000 grams/mole and 155,000 grams/mole.

In a further embodiment, the cellulose-containing feedstock comprises cellulose, hemicellulose, or lignin, or a mixture thereof.

In a preferred embodiment, the cellulose-containing feedstock is selected from woody biomass, pea hulls, corn fiber, soy hulls, citrus residues, cocoa shells, cotton, oat fiber, bran fiber, bagasse, crop grasses, hemp, cereals, fruits, vegetables, paper products and wood products, and mixtures of two or more thereof.

In a preferred embodiment, the first temperature is in the range from 200° C. to less than or equal to 260° C.

In a further preferred embodiment of the present invention, the first time period is in the range from 0.5 second to 4 seconds.

In a further preferred embodiment, the rapidly depressurizing occurs over a period of less than 5% of the first time period.

In a further preferred embodiment of the present invention, the rapid heating occurs over a period of from 5% to 50% of the first time period.

In a further preferred embodiment of the present invention, the first pressure is in the range from 1,500 psi to 3,600 psi.

In a further preferred embodiment of the present invention, the second pressure is in the range of 5 psi to 75 psi.

In a further preferred embodiment of the present invention, the second temperature is in the range from 45° C. to 150° C.

In another embodiment of the present invention, the acid is an inorganic acid, or an organic acid, or a mixture thereof. In a preferred embodiment, the inorganic acid is selected from sulfuric acid, sulfonic acid, phosphoric acid, phosphonic acid, nitric acid, nitrous acid, hydrochloric acid, hydrofluoric acid, hydrobromic acid, and hydroiodic acid, and mixtures thereof. In a further preferred embodiment, the acid is selected from sulfuric acid, nitric acid and hydrochloric acid, and mixtures thereof.

In another embodiment, the organic acid is selected from an aliphatic carboxylic acid, an aromatic carboxylic acid, a dicarboxylic acid, an aliphatic fatty acid, and aromatic fatty acid, an amino acid, an alkyl sulfonic acid, and an acyl sulfonic acid, and mixtures thereof. In another preferred embodiment, the aliphatic carboxylic acid is formic acid or acetic acid or a mixture thereof.

In another preferred embodiment, the aromatic carboxylic acid is selected from benzoic acid and salicylic acid, and mixtures thereof.

In another preferred embodiment the dicarboxylic acid is selected from oxalic acid, phthalic acid, sebacic acid and adipic acid, and mixtures thereof.

In another preferred embodiment, the aliphatic fatty acid is selected from oleic acid, palmitic acid and stearic acid, and mixtures thereof.

In another preferred embodiment, the aromatic fatty acid is phenylstearic acid.

In another preferred embodiment, the alkylsulfonic acid is methane sulfonic acid.

In a further preferred embodiment of the present invention, the pH of the first slurry is in the range from about pH 1.0 to about pH 3.0.

In a further preferred embodiment of the present invention, the cellulose-containing composition prepared by the method of the present invention comprises from 50% to 100% of cellulose based on the total dry weight of the cellulose-containing composition. In a further preferred embodiment of the present invention, the cellulose-containing composition comprises from 60% to 90% of cellulose based on the total weight of the cellulose-containing composition.

In a further preferred embodiment of the present invention, the cellulose-containing composition prepared by the method of the present invention comprises microcrystalline cellulose. In a further preferred embodiment of the present invention, the cellulose-containing composition prepared by the method of the present invention comprises at least 50% microcrystalline cellulose based on the total dry weight of the cellulose-containing composition. In a further preferred embodiment of the present invention, the cellulose-containing composition prepared by the method of the present invention comprises 50% to 90% microcrystalline cellulose based on the total dry weight of the cellulose-containing composition. In a further preferred embodiment of the present invention, the cellulose-containing composition prepared by the method of the present invention comprises at least 75% microcrystalline cellulose based on the total dry weight of the cellulose-containing composition.

In a further preferred embodiment of the present application, the cellulose-containing composition prepared by the method of the present invention comprises a range of microcrystalline cellulose from 80% to 100% microcrystalline cellulose based on the weight of the cellulose in the cellulose-containing composition. In a further preformed embodiment of the present application, the cellulose-containing composition prepared by the method of the present invention comprises a range of microcrystalline cellulose from 90% to 100% microcrystalline cellulose based on the weight of the cellulose in the cellulose-containing composition. In a further preferred embodiment of the present invention, the particles of the microcrystalline cellulose have an aspect ratio of between 2.0 and 10.0. In a further embodiment of the present invention, the particles of microcrystalline cellulose have an aspect ratio of between 2.5 and 8.5.

In a further preferred embodiment of the present application, the cellulose-containing composition prepared by the method of the present invention further comprises lignin, hemicellulose, or a mixture thereof.

In a preferred embodiment, the particles of the cellulose-containing composition prepared by the method of the present invention have a mean particle size between 20 μm and 110 μm.

In a preferred embodiment of the present invention, the separating is accomplished using mechanical separation. In a further preferred embodiment the separating is accomplished using gravity filtration, centrifugal separation, centripetal separation, filtration or a combination thereof.

In another embodiment of the present invention, a cellulose-containing composition prepared by the methods described herein is provided.

In a further embodiment of the present invention, a cellulose-containing composition is provided, comprising from 50% to 100% of cellulose based on the total dry weight of the cellulose-containing composition. In a further embodiment, a cellulose-containing composition is provided, comprising from 60% to 90% cellulose based on the total dry weight of the cellulose-containing composition.

In a further embodiment of the present invention, a cellulose-containing composition is provided, wherein at least a portion of the cellulose-containing composition comprises microcrystalline cellulose. In a preferred embodiment of the present invention, a cellulose-containing composition is provided wherein the cellulose-containing composition comprises at least 50% microcrystalline cellulose based on the total dry weight of the cellulose-containing composition. In a further preferred embodiment of the present invention, a cellulose-containing composition is provided, wherein the cellulose-containing composition comprises from 50% to 90% microcrystalline cellulose based on the total dry weight of the cellulose-containing composition. In a further preferred embodiment, a cellulose-containing composition is provided, wherein the cellulose-containing composition comprises at least 75% microcrystalline cellulose based on the total dry weight of the cellulose in the cellulose-containing composition.

In a preferred embodiment of the present invention, a cellulose-containing composition is provided comprising 80% to 100% microcrystalline cellulose based on the weight of the cellulose in the cellulose-containing composition. In a further preferred embodiment of the present invention, a cellulose-containing composition is provided, comprising a range of microcrystalline cellulose of from 90% to 100% microcrystalline cellulose based on the weight of the cellulose in the cellulose-containing composition.

In a further embodiment of the present invention, a cellulose-containing composition is provided, further comprising lignin and hemicellulose, or a mixture thereof.

In a further embodiment of the present invention, a cellulose-containing composition is provided, wherein the particles of microcrystalline cellulose have an aspect ratio of between 2.0 and 10.0. In a preferred embodiment of the present invention, a cellulose-containing composition is provided, wherein the particles of the microcrystalline cellulose have an aspect ratio of between 2.5 and 8.5.

In a further embodiment of the present invention, a cellulose-containing composition is provided wherein the cellulose contained in the cellulose-containing composition has a weight average molecular weight of between 40,000 grams/mole and 155,000 grams/mole. In a preferred embodiment of the present invention, a cellulose-containing composition is provided wherein the cellulose contained in the cellulose-containing composition has a weight average molecular weight of between 75,000 grams/mole and 155,000 grams/mole.

In a further embodiment of the present invention, a cellulose-containing composition is provided, wherein the particles of the cellulose-containing composition have a mean particle size between 20 μm and 110 μm.

Suitable cellulose-containing feedstocks include, but are not limited to, wood products, pea hulls, corn fiber, soy hulls, citrus residues, cocoa shells, cotton, oat hull fiber, bran fiber, bagasse, crop grasses, hemp, cereals, fruits, vegetables, and paper products and mixtures thereof.

A further embodiment of the present invention provides a method for the preparation of a cellulose-containing composition, comprising the steps of:
(a) forming a first slurry comprising a cellulose-containing feedstock, water, and acid;
(b) subjecting the first slurry to rapid heating to a first temperature, by addition of subcritical, near critical or supercritical water, at a first pressure, thereby forming a second slurry;
(c) maintaining the second slurry at the first temperature and first pressure for a first time period; and
(d) rapidly depressurizing the second slurry to form a third slurry at a second temperature and a second pressure;
(e) concentrating the third slurry to form a cellulose-containing composition comprising the dissolved solids of the third slurry, and the suspended solids of the third slurry;
wherein the first temperature is in the range of 180° C. to 280° C.; and
   wherein the cellulose, comprising the cellulose-containing composition comprising the dissolved solids of the third slurry, and the suspended solids of the third slurry, has a weight average molecular weight between 40,000 grams/mole and 155,000 grams/mole.

In a further embodiment of the present invention, the concentrating of the third slurry is performed by filtration of the third slurry to form a residue and a filtrate, concentrating the filtrate of the third slurry, and adding the concentrated filtrate back into the residue. In a further embodiment of the present invention, the concentrating of the third slurry is performed by centrifugation, to form a pellet and a supernatant, the supernatant is concentrate, and then added back to the pellet. In a further embodiment of the present invention, the concentrating of the third slurry is performed by concentration of the third slurry in vacuo.

In a further embodiment of the present invention, the cellulose contained in the cellulose-containing composition comprising the dissolved solids of the third slurry, and the suspended solids of the third slurry, has a weight average molecular weight between 75,000 grams/mole and 155,000 grams/mole.

In a further embodiment, the cellulose-containing feedstock used to prepare the cellulose-containing composition wherein the cellulose-containing composition comprising the dissolved solids of the third slurry, and the suspended solids of the third slurry, comprises cellulose, hemicellulose, or lignin, or a mixture thereof.

In a further embodiment of the present invention, the cellulose-containing feedstock used to prepare the cellulose-containing composition, comprising the cellulose-containing composition comprising the dissolved solids of the third slurry, and the suspended solids of the third slurry, is selected from wood products, pea hulls, corn fiber, soy hulls, citrus residues, cocoa shells, cotton, oat fiber, bran fiber, bagasse, crop grasses, hemp, cereals, fruits, vegetables, paper products and wood products, and mixtures thereof.

In a preferred embodiment, the first temperature in the method for the preparation of the cellulose-containing composition comprising the dissolved solids of the third slurry, and the suspended solids of the third slurry is in the range from 200° C. to less than or equal to 260° C.

In another preferred embodiment of the present invention, the first time period in the method for the preparation of the cellulose-containing composition, wherein the cellulose-containing composition comprises the dissolved solids of the third slurry, and the suspended solids of the third slurry, is from 0.5 seconds to 4 seconds.

In another embodiment of the present invention, the rapidly depressurizing in the method for preparation of the cellulose-containing composition, wherein the cellulose-containing composition comprises the dissolved solids of the third slurry, and the suspended solids of the third slurry, occurs over a period of less than 5% of the first time period.

In a further embodiment of the present invention, the rapid heating in the method for the preparation of the cellulose-containing composition, wherein the cellulose-containing composition comprises the dissolved solids of the third slurry, and the suspended solids of the third slurry, occurs over a period of from 5% to 50% of the first time period.

In another embodiment of the present invention, the first pressure of the method for the preparation of the cellulose-containing composition, wherein the cellulose-containing composition comprises the dissolved solids of the third slurry, and the suspended solids of the third slurry, is in the range of 1,500 psi to 3,600 psi.

In a further embodiment of the present invention, the second pressure of the method for the preparation of the cellulose-containing composition, wherein the cellulose-containing composition comprises the dissolved solids of the third slurry, and the suspended solids of the third slurry, is in the range of 5 psi to 75 psi.

In another embodiment of the present invention, the second temperature of the method for the preparation of the cellulose-containing composition, wherein the cellulose-containing composition comprises the dissolved solids of the third slurry, and the suspended solids of the third slurry, is in the range from 45° C. to 150° C.

In a further embodiment of the present invention, the acid of the method for the preparation of the cellulose-containing composition, wherein the cellulose-containing composition comprises the dissolved solids of the third slurry, and the suspended solids of the third slurry, is an organic acid, or an inorganic acid, or a mixture thereof. In a preferred embodiment, the inorganic acid is selected from sulfuric acid, sulfonic acid, phosphoric acid, phosphonic acid, nitric acid, nitrous acid, hydrochloric acid, hydrofluoric acid, hydrobromic acid, and hydroiodic acid, and mixtures thereof. In a further preferred embodiment, the acid is selected from sulfuric acid, nitric acid and hydrochloric acid and mixtures thereof. In another preferred embodiment, the organic acid is selected from an aliphatic carboxylic acid, an aromatic carboxylic acid, a dicarboxylic acid, an aliphatic fatty acid, an aromatic fatty acid, an amino acid, an alkyl sulfonic acid, and mixtures thereof. In a preferred embodiment, the aliphatic carboxylic acid is formic acid or acetic acid, or a mixture thereof. In a further preferred embodiment, the aromatic carboxylic acid is selected from benzoic and salicylic acid, and mixtures thereof. In a further preferred embodiment, the aliphatic acid is selected from oleic acid, palmitic acid and stearic acid, and mixtures thereof. In a further preferred embodiment, the aromatic fatty acid is phenylstearic acid. In a further preferred embodiment, the alkyl sulfonic acid is methane sulfonic acid.

In a further embodiment, the pH of the first slurry in the method for preparation of the cellulose-containing composition, wherein the cellulose-containing composition comprises the dissolved solids of the third slurry, and the suspended solids of the third slurry, is from pH 1.0 to pH 3.0.

In a further embodiment of the present invention, a cellulose-containing composition is provided, wherein the cellulose-containing composition comprises the dissolved solids of the third slurry, and the suspended solids of the third slurry, obtained by the methods of preparation of the cellulose-containing composition, wherein the cellulose-containing composition comprises the dissolved solids of the third slurry, and the suspended solids of the third slurry, according to any of the foregoing embodiments.

In a further embodiment of the present invention, a cellulose-containing composition is provided, wherein the cellulose-containing composition comprises the dissolved solids of the third slurry, and the suspended solids of the third slurry, comprising a soluble glucose monomer, a soluble glucose oligomer, a soluble xylose monomer, a soluble xylose oligomer, or a combination thereof. In a further embodiment of the present invention, a cellulose-containing composition is provided, wherein the cellulose-containing composition comprises the dissolved solids of the third slurry, and the suspended solids of the third slurry, comprising water, so as to provide a moisture content of from 5 wt % to 90 wt %. In a further embodiment of the present invention, a cellulose-containing composition is provided, wherein the cellulose-containing composition comprises the dissolved solids of the third slurry, and the suspended solids of the third slurry, comprising from 25% to 50% of cellulose, based on the total dry weight of the cellulose-containing composition. In a further embodiment of the present invention, a cellulose-containing composition is provided, wherein the cellulose-containing composition comprises the dissolved solids of the third slurry, and the suspended solids of the third slurry, comprising from 40% to 50% of cellulose, based on the total dry weight of the cellulose-containing composition. In a further embodiment of the present invention, a cellulose-containing composition is provided, wherein the cellulose-containing composition comprises the dissolved solids of the third slurry, and the suspended solids of the third slurry, wherein at least a portion of the cellulose-containing composition comprises microcrystalline cellulose. In a further embodiment of the present invention, a cellulose-containing composition is provided, wherein the cellulose-containing composition comprises the dissolved solids of the third slurry, and the suspended solids of the third slurry, comprising at least 25% microcrystalline cellulose based on the total dry weight of the cellulose-containing composition. In a further embodiment of the present invention, a cellulose-containing composition is provided, wherein the cellulose-containing composition comprises the dissolved solids of the third slurry, and the suspended solids of the third slurry, comprising a range of between 25% microcrystalline cellulose to 50% microcrystalline cellulose based on the total dry weight of the cellulose-containing composition.

In a further embodiment of the present invention, a cellulose-containing composition is provided, wherein the cellulose-containing composition comprises the dissolved solids of the third slurry, and the suspended solids of the third slurry, comprising at least 75% microcrystalline cellulose based on the weight of the cellulose in the cellulose-containing composition. In a further embodiment of the present invention, a cellulose-containing composition is provided, wherein the cellulose-containing composition comprises the dissolved solids of the third slurry, and the suspended solids of the third slurry, comprising a range of from 80% to 100% microcrystalline cellulose based on the weight of the cellulose in the cellulose-containing composition. In a further embodiment of the present invention, a cellulose-containing composition is provided, wherein the cellulose-containing composition comprises the dissolved solids of the third slurry, and the suspended solids of the third slurry, comprising a range of from 90% to 100% microcrystalline cellulose, based on the weight of the cellulose in the cellulose-containing composition. In a further embodiment of the present invention, a cellulose-containing composition is provided, wherein the cellulose-containing composition comprises the dissolved solids of the third slurry, and the suspended solids of the third slurry, wherein the cellulose-containing composition further comprises lignin, hemicellulose, or a mixture thereof.

In a further embodiment of the present invention, a cellulose-containing composition is provided, wherein the cellulose-containing composition comprises the dissolved solids of the third slurry, and the suspended solids of the third slurry, wherein the particles of microcrystalline cellulose have an aspect ratio of between 2.0 and 10.0. In a further embodiment of the present invention, a cellulose-containing composition is provided, wherein the cellulose-containing composition comprises the dissolved solids of the third slurry, and the suspended solids of the third slurry, wherein the particles of microcrystalline cellulose have an aspect ratio of between 2.5 and 8.5. In a further embodiment of the present invention, a cellulose-containing composition is provided, wherein the cellulose-containing composition comprises the dissolved solids of the third slurry, and the suspended solids of the third slurry, wherein the cellulose contained in the cellulose-containing composition has a weight average molecular weight between 40,000 grams/mole and 155,000 grams/mole. In a further embodiment of the present invention, a cellulose-containing composition is provided, wherein the cellulose-containing composition comprises the dissolved solids of the third slurry, and the suspended solids of the third slurry, wherein the cellulose contained in the cellulose-containing composition has a weight average molecular weight between 75,000 grams/mole and 125,000 grams/mole. In a further embodiment of the present invention, a cellulose-containing composition is provided, wherein the cellulose-containing composition comprises the dissolved solids of the third slurry, and the suspended solids of the third slurry, wherein the particles of the cellulose-containing composition have a mean particle size between 20 μm and 110 μm.

The cellulose-containing compositions of the present invention, may be provided as compositions comprising as little as about 5 wt % water, or as slurries comprising up to about 90 wt % water. How the compositions are optimally provided may depend on a number of factors, including, for example, ease of shipment of the composition, suitability for a final use of the composition, length of storage of the composition, and the like.

### Definitions

As used herein, “aspect ratio” refers to the ratio of the largest dimension of a particle to the smallest (e.g. length/diameter for a cylinder; length/thickness for a plate; longest axis/shortest axis for an ellipsoid).

As used herein, “cellulose-containing feedstock” means material comprising carbon-based biological material derived from living or recently-living organisms, or recovered or upcycled from materials or products comprising them, for example, plants, trees, dedicated energy crops, agricultural products and agricultural by-products, and may include paper products or wood products, at least of portion of which is cellulose.

As used herein, “continuous” indicates a process which is uninterrupted for its duration or, if interrupted, paused or suspended only momentarily relative to the duration of the process. Treatment of the cellulose-containing feedstock is “continuous” when the cellulose-containing feedstock is fed into the apparatus without interruption or without a substantial interruption, or processing of said cellulose-containing feedstock is not done in a batch process.

As used herein, the terms “microcrystalline cellulose” or “MCC” refers to purified, partially depolymerized cellulose prepared by hydrolysis of cellulose fibers. Cellulose fiber typically comprises cellulose microfibers comprising amorphous, paracrystalline, and crystalline regions. The hydrolysis process largely removes the amorphous fraction, destroying the fiber-like morphology of the cellulose and forming the cellulose microcrystals containing wholly or mostly crystalline regions. Commercially available MCC includes, but is not limited to, AVICEL® products available from FMC BioPolymer. Traditional methods of preparation of MCC involve concentrated acid for prolonged periods of time. The methods of the present invention use much lower concentrations of acid for very short time periods.

As used herein, an “oligomer” has a degree of polymerization of 2 to about 9, for example the number of sugar monomers contained in a given oligomer. Sugar monomers include, for example, those of glucose, xylose, mannose, fructose and arabinose

As used herein, “resides” or “residence time” indicates the length of time which a given portion or bolus of material is within a reaction zone or reactor vessel.

A supercritical fluid is a fluid at a temperature above its critical temperature and at a pressure above its critical pressure. A supercritical fluid exists at or above its “critical point,” the point of highest temperature and pressure at which the liquid and vapor (gas) phases can exist in equilibrium with one another. Above critical pressure and critical temperature, the distinction between liquid and gas phases disappears. A supercritical fluid possesses approximately the penetration properties of a gas simultaneously with the solvent properties of a liquid. Accordingly, supercritical fluid extraction has the benefit of high penetrability and good solvation.

As used herein, “supercritical water” means water, at or above a critical temperature of about 374.2° C., and a critical pressure of about 221 bar. “Near critical water” has a temperature at or above about 300° C. and below the critical temperature of water (374.2° C.), and at a pressure high enough to ensure that all fluid is in the liquid phase. Sub-critical water has a temperature of less than about 300° C. and a pressure high enough to ensure that all fluid is in the liquid phase. Sub-critical water temperature may be greater than about 250° C. and less than about 300° C., and in many instances sub-critical water has a temperature between about 250° C. and about 280° C.

As used herein, the term “weight-average molecular weight” (Mw) is defined by the formula:

$${M}_{w} = \frac{\sum {N}_{i} {M}_{i}^{2}}{\sum {N}_{i} {M}_{i}} ,$$

where Mᵢ is the molecular weight of a chain and Ni is the number of chains of that molecular weight.

In the following portions of this disclosure, headed, “Thickeners”, “Suspensions”, “Emulsions”, “Food Products”, and “Personal Care and Cosmetic Products”, unless otherwise indicated, the phrase “Cellulose-Containing Compositions” refers to either or both of (1) the cellulose-containing composition of the present invention, wherein the cellulose-containing composition comprises the solid portion of the third slurry as disclosed herein, and (2) the cellulose-containing composition of the present invention, wherein the cellulose-containing composition comprises both suspended solids of the third slurry, and the dissolved solids of the third slurry as disclosed herein.

### Thickeners

The Cellulose-Containing Compositions of the present invention can exist as a stable suspension in water (or other suitable liquid or liquid mixture) and also can provide favorable rheological properties, such as thixotropy, which is usually considered to be desirable in coatings applications, such as architectural and industrial paints (it allows relatively facile brush, roller, or spray application of the paint, without the paint running or dripping from the applied surface).

In various aspects, the Cellulose-Containing Compositions of the present invention as disclosed herein, are present in a thickened composition or formulation in an amount, (based on the total weight of the thickened composition or formulation) of 0.05% to 15%.

In one aspect, thickened compositions are contemplated comprising the Cellulose-Containing Compositions of the present invention, and comprising a liquid; wherein the cellulose particles are present at a level sufficient to increase the viscosity of the composition or liquid by at least 10% compared to an otherwise identical composition without the cellulose particles.

In various aspects, the compositions further comprise one or more additional thickeners, such that the resulting viscosity is greater than the sum of (a) the viscosity of the otherwise identical compositions with the Cellulose-Containing Compositions of the present invention, and (b) the viscosity of the otherwise identical compositions with the one or more additional thickeners without the Cellulose-Containing Compositions of the present invention. Suitable additional thickeners include starch (e.g., arrowroot, cornstarch, katakuri starch, potato starch, sago, tapioca, and derivatives thereof), vegetable gum (e.g., alginin, guar gum, locust bean gum, xanthan gum, and derivatives thereof, such as hydroxypropyl guar), proteins (e.g., collagen, egg whites, gelatin, cassein, and derivatives thereof), modified castor oil, organosilicones (silicone resins, dimethicones), saccharides and polysaccharides (pectin, agar, carrageenan, pullulan, konjac, and alginate), polyurethanes (such as HEUR thickeners: hydrophobically modified ethylene oxide based urethane thickeners), acrylic polymers (e.g., polyacrylic acid, polymethacrylic acid, and copolymers of acrylic acid or methacrylic acid with acrylic monomers such as alkyl acrylate, alkyl alkylacrylate, for example, methyl acrylate, methylmethacrylate, etc. (where “alkyl” is any C1-C5 group)), latex polymers, styrene/butadiene, polyvinyl alcohol, clay (e.g., attapulgite, bentonite, montmorillonite), modified cellulose (methylhydroxyethyl cellulose, carboxymethyl cellulose, hydroxymethyl cellulose, hydroxypropyl methyl cellulose), polyethylene glycol, petroleum jelly, wax, silica (e.g., fumed silica, colloidal silica, hydrated silica), or any combination thereof.

In various aspects, the thickened composition contemplated further comprises other formulation ingredients, or a combination of ingredients, depending on the use of the thickened composition. For example, in various aspects, the thickened composition further comprises pigment particles, filler or extender particles, polymer particles, or a combination thereof, and/or other formulation ingredients. In various aspects, the thickened composition is a paint, coating, ink, adhesive or sealant. In various aspects, the thickened composition may be a personal care product, for example, health and beauty or cosmetic product, such as, for example, a lotion, cream, ointment, serum, shampoo, conditioner, hairspray, hair gel, deodorant, facial or body wash, facial or body scrub, exfoliant, emollient, moisturizer, soap, foundation make up, BB cream, CC cream, eye cream, sunscreen, anti-acne serum or cream or lotion, cellular serum or cream or lotion, facial or body mask, blush, eyeshadow, mascara, lipstick or lip balm, or clay, kaolin or mud suspension.

In various aspects, the thickened composition is a sunscreen containing a light absorbing compound, a light scattering compound, or a combination thereof. The light absorbing or light scattering compound can be, e.g., an oxide particle (e.g., zinc oxide, titanium oxide), a polymer particle (such as hollow sphere pigment, comprising polystyrene, or acrylic or styrene-acrylic copolymers), an organic compound (p-aminobenzoic acid, octyldimethyl p-aminobenzoic acid, phenylbenzimidazole sulfonic acid, cinoxate, dioxybenzone, oxybenzone, homosalate, menthyl anthranilate, octocrylene, octyl methoxycinnamate, octyl salicylate, sulisobenzone, trolamine salicylate, avobenzone, ecamsule, 4-methylbenzylidene, bisoctrizole, bemotrizinol, tris-biphenyl triazine, bisimidazylate, drometrizole trisiloxane, sodium dihydroxyl dimethoxy disulfobenzopenone, octyl triazone, diethylamino hydroxybenzoyl hexyl benzoate, iscotrizinol, dimethico-diethylbenzalmalonate, amiloxate), or any combination thereof. For some uses, the higher viscosity of the thickened composition is desirable to avoid any running or dripping of the product either during application of the product to the surface or running or dripping of the product from the applied surface. In various aspects, the thickened composition may be more aptly characterized as a suspension when the thickened composition contains a suspended particle, such as an inorganic particle or polymer particle, or as an emulsion.

In various aspects, the thickened composition may be a cleaning fluid, such as, for example, dishwashing detergent, laundry detergent, liquid fabric conditioner, no-splash bleach, toilet bowl cleaner, or drain cleaner, or industrial detergents or cleaning fluids. Higher viscosity is desirable in these thickened cleaning compositions so that, for example, the cleaning product does not run straight off the surface (e.g., in a toilet bowl), but clings long enough for some adsorption and cleaning effect to occur. In some cases, it is also helpful to have a higher viscosity to minimize splashing of the liquid composition, especially for types of cleaning fluids comprising harsh chemicals that may be skin or eye or respiratory irritants (such as, e.g., sodium hypochlorite in household bleach). In various aspects, such as in drain cleaners, the higher viscosity is desirable to allow the thickened composition to remain cohesive (i.e., stay together) and to sink when added to water to allow the composition to reach and act on the drain clog.

In various aspects, the thickened composition may be an edible composition, such as, for example, a beverage, smoothie, shake, syrup, soup, broth, sauce, marinade, dressing, gravy, pie filling, condiment, pudding, or pet food or treat. The benefits of higher viscosity for some food products can relate to texture and mouth-feel and to convenience of handling.

### Suspensions

In one aspect of the present invention, suspensions are contemplated comprising the Cellulose-Containing Compositions of the present invention and a first component suspended within the composition.

The Cellulose-Containing Compositions disclosed herein, provide stabilization to solid particles in suspension. In various aspects, the suspension is a stable suspension. For solid particles that do not dissolve in solution (e.g., aqueous solution), and do not form a stable or metastable suspension, but instead, over a shorter or longer time period, settle on the bottom of the container (e.g., calcium carbonate), or even float to the surface (e.g., cocoa powder and cinnamon both settle out and has some portion rise to the surface), the Cellulose-Containing Compositions disclosed herein can function as a suspension aid and allow the formation of homogeneous stable or metastable suspensions. In certain end uses, even temporary stabilization of solid particles in a suspension may be beneficial. For example, a solid may need to only be temporarily suspended (e.g., on the order of minutes) to, for example, enable pumping. In various aspects, the cellulose-containing compositions herein function as a suspension aid and allow the formation of metastable suspensions, such as, for example a 50-95% stable suspension. The mechanism of stabilization is not known, although, without wishing to be bound by theory, in some case the cellulose-containing compositions may provide improved rheology (e.g., higher viscosity) which may contribute to the stabilization of a first component solid in suspension. In various aspects, the suspension comprising the Cellulose-Containing Compositions of the present invention are sufficiently thickened to enable the first component to be in a stable suspension in the composition. In some aspects, the formation of an emulsion facilitates suspension of a solid.

The Cellulose-Containing Compositions described herein can provide stabilization of a first component in a composition. The first component can be a solid within the composition. The first component typically is insoluble, or at least partially insoluble, in the composition at ambient conditions at the concentration that the first component is intended to be employed in the compositions. For example, the first component can be a plurality of pigment particles (e.g., titanium dioxide, red iron oxide, yellow iron oxide, black iron oxide, umber, cobalt violet, ultramarine, cadmium green, chrome green, cadmium orange, red ochre, yellow ochre, carbon black, barium sulfate, and the like, or any combination thereof), filler/extender particles (e.g., calcium carbonate, mica, wollastonite, amorphous silica, and the like, or any combination thereof), polymer particles (e.g., polyethylene, polypropylene, polystyrene, Kevlar™, and the like, or any combination thereof), inorganic particles (e.g., silica, alumina, titania, iron oxide, zinc oxide, magnesium silicate, clay, calcium carbonate, or the like, or any combination thereof), ore or mineral particles (e.g., iron ore, bauxite, hematite, calcium phosphate), coal dust or charcoal particles, dirt particles, rock cuttings, cocoa particles, an active pharmaceutical ingredient, an excipient, an exfoliant, a fiber, or any combination thereof. Specific non-limiting examples of first components and the compositions containing them include use in various industrial uses, including stabilization of solids such as: pigment particles, filler/extender particles or polymer particles in paints, coatings, caulks, sealants or adhesives; inorganic particles in cement or concrete; dirt particles or rock cuttings in drilling mud or drilling fluid; ore or mineral particles in a mining slurry; coal dust or charcoal particles in a fuel slurry; solid particles (such as molybdenum disulfide or a fluoropolymer) in a solid lubricant or grease; and oxide particles in a cleaning or buffing slurry or polish (such as a car detailing cleaner, or a chemical mechanical planarization slurry). Specific non-limiting examples of first components and the compositions containing them include use in various pharmaceutical, or personal care or beauty products, and health products, including stabilization of solids such as: an excipient or an active pharmaceutical ingredient in a medicine or supplement; polymer particles or inorganic particles or oxide particles (e.g., hollow sphere pigment, titanium dioxide or zinc oxide) in a lotion, sunscreen, BB cream, CC cream; pigment particles or inorganic particles in a make-up foundation, blush, eye shadow, mascara, facial or body mask, or clay/kaolin/mud suspension; exfoliant particles (e.g., the polymer particles or inorganic particles as disclosed herein, or any combination thereof) in a skin care product or scrub; inorganic or polymer particles in a soap; and inorganic or polymer particles in a toothpaste. Specific non-limiting examples of first components and the compositions containing them include use in various food or beverage products, including stabilization of solids such as: cocoa, malt, or artificially or naturally flavored particles (including herbs and spices) in a milk, hot or cold beverage, syrup, dressing, marinade, soup, or sauce; fruit, vegetable, fiber or protein particles in a beverage, smoothie, or shake, and pet food or pet treat.

In various aspects, the Cellulose-Containing Compositions of the present invention are present in a liquid suspension of at least partially insoluble particles in an amount (weight %, cellulose-containing composition of the present invention based on the total weight of the suspension) of from 0.05% to 15%.

### Emulsions

The Cellulose-Containing Compositions of the present invention can stabilize oil/fluid emulsions, for example, Pickering emulsions of oil and water.

In various aspects, the emulsion or emulsifiable composition is an emulsion, and the emulsion comprises: a first fluid and a second fluid; wherein the first fluid is at least partially immiscible with the second fluid.

In a further aspect, the first fluid comprises an oil and the second fluid comprises water. Suitable liquids that are immiscible with water include olive oil, canola oil, vegetable oil, coconut oil, peanut oil, corn oil, palm oil, palm kernel oil, or any combination thereof. In various aspects, the emulsion or emulsifiable composition is an egg-free composition.

In a further aspect, the emulsion is a non-food related emulsion. For example, an emulsion can be used to formulate a personal care product (e.g., acne gels). In a further example, an emulsion can be used as an industrial formulation (e.g., paint).

In a further aspect, the second fluid comprises an oil and the second fluid comprises vinegar. Accordingly, the Cellulose-Containing Compositions of the present invention may find use in any applications for which formulations comprising two at least partially immiscible liquids (e.g., a hydrophobic liquid and a hydrophilic liquid) are useful, particularly oil/water emulsions. Accordingly, in various aspects, the emulsion is, or is a component of, a mayonnaise, a salad dressing, a sandwich spread, vegetable spread, vegetable shortening, a vinaigrette, a condiment, a cheese, a yogurt, an ice cream, a sauce, a butter, a nut butter, a margarine, a cream, a milk, a gravy, a coffee beverage, chocolate, or a sauce comprising oil/water emulsions (such as, e.g., pasta sauces, BBQ sauces, hot sauces, hollandaise, béarnaise, etc.), as well as baked goods that utilize an oil/water emulsion in the pre-baked formulation. In various aspects, the emulsion is, or is a component of, a fruit-butter, -sauce, -jelly, -jam, -chutney, -custard, -marinade, or -soup. In some embodiments, the emulsion comprising the cellulose-containing compositions of the present invention is, or is a component of, pet food or pet treats.

In various aspects, the Cellulose-Containing Compositions described herein are present in emulsions in an amount (weight % of the Cellulose-Containing Compositions described herein based on the total weight of the emulsion formulation) of from 0.05% to 15.0%.

In various aspects, the Cellulose-Containing Compositions described herein are present in emulsifiable compositions, for example in pre-mix formulations wherein the emulsifiable composition or formulation is, or is a component of, a mayonnaise mix, a salad dressing mix, a sandwich spread mix, vegetable spread mix, a vinaigrette mix, a milk mix, a gravy mix, or a coffee beverage mix, or a sauce mix (e.g. pasta sauce, BBQ sauce, hot sauce and the like). In various aspects, the emulsifiable composition is in a powder form, a granular form, a paste, or a concentrate. Such mixes are contemplated to be those that are packaged by a manufacturer of mixes to be prepared into a vinaigrette, mayonnaise, dressing, etc., by a third party, whether it be an individual consumer at home, an industrial producer, or a restaurant, caterer, or other food preparer of such vinaigrettes, mayonnaises, pet food (e.g., a “gravy” that forms when you pour water over the dry food), etc.

In various aspects, the particles comprising the Cellulose-Containing Compositions described herein are present in emulsifiable compositions in an amount (weight % of the cellulose-containing composition based on the total weight of the emulsifiable composition or formulation) of from 0.05% to 40%.

Furthermore, the Cellulose-Containing Compositions disclosed herein may find use in stabilizing emulsions, or a component of the emulsions, in any application for which at least partially immiscible liquids are used together in a composition, particularly oil/water emulsions, and may be used in the same amounts as disclosed above concerning compositions and formulations comprising two immiscible liquids. Such applications include, for example, pharmaceuticals (the active ingredient may be a liquid that is immiscible in water, but more conveniently delivered in aqueous carrier formulation); personal care or health and beauty products such as a cosmetic product (e.g., foundation make up, a lipstick or lip balm, a mascara, blush, eyeshadow), a skin-care product, such as creams (e.g., hand, face, and/or body creams and moisturizers/emollients, bb cream, cc cream, eye cream, anti-acne serum or cream, cellular serum or cream), lotions or ointments (e.g., dermatological lotions or ointments, such as, e.g., anti-acne lotion or cellular lotion), sunscreen products, or a hair care product (such as hair conditioners, shampoo, hair gel, hairspray, or hair dye), or a toothpaste, teeth whitener, or fluoride composition; agricultural products, such as, for example, delivery vehicles for pesticides, insecticides, biocides, fungicides, herbicides and fertilizers; waterborne coatings wherein the hydrophobic polymer is stabilized in emulsion form (e.g., which has been formed via inversion by addition of water into the resin), such as, for example, waterborne alkyd resin, polyester resin, epoxy resin, acrylic resin, polyurethane, fluoropolymer, wax emulsion, etc.); industrial chemical additives, such as, for example, silicone defoamers, biocides and colorants used in formulations like paints, coatings, sealants, caulks and inks; drilling fluids, for example as used in oil wells. In various aspects, the emulsion is, or is a component of, a subterranean treatment composition (such as a drilling fluid), or is created in situ when such composition or drilling fluid is used in treating or drilling; or a metalworking fluid or a component of a metalworking fluid, or is created in situ when a metalworking fluid is used in metalworking; or a cutting fluid, stamping fluid, abrading fluid, tribological fluid, cooling fluid, or lubricating fluid, or component thereof, or is created in situ when such fluids are used in cutting, stamping, abrading, tribological modification, cooling, or lubricating, respectively. In various aspects, the emulsion is, or is a component of, a leather care product or shoe polish. The Cellulose-Containing Compositions disclosed herein may also find use (and at the same amounts disclosed above) in any formulation that functions at least in part by stabilizing an immiscible liquid in a carrier liquid, even temporarily, such as, for example, laundry detergents, dishwasher fluids or solids, dry-cleaning formulations, industrial detergents, etc. Accordingly, in various aspects, the emulsion is, or is a component of a cleaning agent, dishwasher fluid, dishwasher paste, laundry detergent, laundry paste, liquid fabric conditioner, no-splash bleach, toilet bowl cleaner or drain cleaner, a dry-cleaning cleaning formulation, or industrial cleaning fluids and detergents.

In various aspects, the emulsion may be an edible composition, such as, for example, a beverage, smoothie, shake, syrup, soup, broth, sauce, marinade, dressing, gravy, pie filling, condiment, pudding, or pet food or treat. The higher viscosity for some food products often relates to texture and mouth-feel, but convenience of handling is another factor.

### Food Products

In various aspects, food products comprising the Cellulose-Containing Compositions of the present invention, are contemplated.

Cooked or baked goods that utilize an oil/water emulsion in the pre-baked formulation include leavened or leavenable food products, such as breads, cakes, sponge puddings and various baked goods, which may include, for example, muffins, brownies, pasta, etc.

Many cooked or baked goods include egg in the formulation; the egg yolk provides lecithin, which acts as an emulsifier. As described herein, the Cellulose-Containing Compositions of the present invention, are also able to stabilize oil/water emulsions. The egg white coagulates as it cooks and, as such, provides structure to baked goods. The Cellulose-Containing Compositions disclosed herein help provide this structural integrity, cohesive strength and elasticity to cakes, breads, and other baked goods.

Thus, in one aspect of the present invention, contemplated are leavened or leavenable food products comprising the Cellulose-Containing Compositions of the present invention.

In various aspects, the leavened or leavenable food product is a leavened food product, and the leavened food product is a bagel, a muffin, a scone, a bread, a pizza base, a cracker, a pastry, a pie, a cake, a shortcake, a cupcake, a pancake, a waffle, a sponge pudding, a Yorkshire pudding, a doughnut, a bun, a brownie, a blondie, a biscuit, a cookie, a pasta, a noodle, pet food, or pet treats.

In various aspects, the Cellulose-Containing Compositions described herein are present in a cooked or baked food product, such as a leavened food product in an amount (weight %, dry solids of the cellulose-containing compositions of the present invention based on the total weight of the cooked or baked food product on a dry basis) of 0.05% to 15%.

In various aspects, the leavened or leavenable food product is a leavenable food product, and the leavenable food product is a bagel mix, a muffin mix, a scone mix, a bread mix, a pizza base mix, a cracker mix, a pastry mix, a pie mix, a cake mix, a shortcake mix, a cupcake mix, a pancake mix, a waffle mix, a sponge pudding mix, a Yorkshire pudding mix, a doughnut mix, a bun mix, a brownie mix, a blondie mix, a biscuit mix, a cookie mix, a pasta mix, a noodle mix, a flour composition, or a dough thereof.

In various aspects, the Cellulose-Containing Compositions described herein are present in an uncooked food formulation, such as a leavenable food product, cookie dough, bread dough, pizza base dough, bun dough, doughnut dough, pasta dough, or a batter or dry or partially dry mix for a bagel, muffin, scone, bread, pizza base, cracker, pastry, pie, cake, shortcake, cupcake, pancake, waffle, sponge pudding, Yorkshire pudding, doughnut, bun, brownie, blondie, biscuit, cookie, pasta, noodle, or the like in an amount (weight %, dry solids of particles comprising cellulose based on the total weight on a dry basis of the pre-cooked or uncooked formulation) of 0.05% to 40%.

In various aspects, the Cellulose-Containing Compositions described herein are present in an uncooked food formulation, such as a leavenable food product, cookie dough, bread dough, pizza base dough, bun dough, doughnut dough, pasta dough, or a batter or dry or partially dry mix for a bagel, muffin, scone, bread, pizza base, cracker, pastry, pie, cake, shortcake, cupcake, pancake, waffle, sponge pudding, Yorkshire pudding, doughnut, bun, brownie, blondie, biscuit, cookie, pasta, noodle, or the like in an amount (weight % of the Cellulose-Containing Compositions based on the total weight on a dry basis of the pre-cooked or uncooked formulation) of 0.05% to 40%.

Furthermore, the Cellulose-Containing Compositions of the present invention can also stabilize gas bubbles-foam in compositions and provide structure, cohesive strength and elasticity in leavened breads, baked goods, and pasta. Such properties are often provided by gluten from the flour in these baked goods; accordingly, the compositions of the present invention are also able to support structure development in gluten-free compositions, and so may find utility in replacing gluten in these systems (not necessarily a direct 1:1 by weight replacement), thereby allowing formulation of gluten-free baked goods. Since a sub-set of the population has an allergy to eggs, and another subset of the population (overlapping or not) has an allergy to gluten, baked goods that are both egg-free and gluten-free are sometimes referred to as allergen-free. Advantageously, if viewed in this way, the Cellulose-Containing Compositions of the present invention may find utility in the formulation and production of egg-free, gluten-free, or allergen-free foodstuffs, such as egg-free, gluten-free, or allergen-free bread, muffins, cakes, brownies, pasta, etc. In addition, there is a strong and growing market trend to eat “gluten-free” and/or “vegan,” which can be satisfied by utilizing the Cellulose-Containing Compositions of the present invention.

In various aspects, the leavened or leavenable food product comprising the Cellulose-Containing Compositions disclosed herein is egg-free. In various aspects, the leavened or leavenable food product comprising the Cellulose-Containing Compositions disclosed herein is gluten-free. In various aspects, the leavened or leavenable food product comprising the Cellulose-Containing Compositions disclosed herein is egg-free and gluten-free. In various aspects, the leavened or leavenable food product comprising the Cellulose-Containing Compositions disclosed herein is allergen-free.

The Cellulose-Containing Compositions disclosed herein also find use as an ingredient in ice cream. Ice cream requires the co-stabilization of ice crystals, air bubbles and fat droplets from the cream, together with an aqueous sugar solution. Ice cream thus contains all three states of matter simultaneously, and is both a foam and an oil in water emulsion. As disclosed herein, the Cellulose-Containing Compositions can stabilize air bubbles and also stabilize oil in water emulsions. Ice cream prepared using the Cellulose-Containing Compositions disclosed herein does not require any unnatural emulsifiers often found in commercial ice cream, or egg component as found in some custard-based ice creams. Ice cream prepared containing the Cellulose-Containing Compositions of the present invention may also melt more slowly than a conventional ice cream. This has importance for the end-user, for example, as the ice cream is being eaten from a cone, so that it does not drip on one's clothes. It also has importance during transportation outside of a refrigerated truck, either from the point of manufacture to the store, or from the store to the consumer household. Any melting that occurs at those times affects the ice crystal size and the preferred (small) ice crystal size cannot be recovered simply by re-freezing at the point of destination. The creamy quality of the ice cream is irreversibly and negatively impacted by such melting.

In another aspect, the amount of fats (in the form of oils or butter) can be reduced in many leavened and leavenable products when adding the Cellulose-Containing Compositions of the present invention, which in turn reduces the calorie content of the baked good.

The Cellulose-Containing Compositions of the present invention can aid in maintaining moisture content during baking and increasing shelf-life.

The Cellulose-Containing Compositions of the present invention may also be used as fiber additives in food products, and as fillers in food products.

Compositions containing the Cellulose-Containing Compositions of the present invention have been found to support stable air-in-water foams. Typically, in order to form a stable foam, a surfactant such as lecithin, mono-glycerides or proteins, must be present to reduce the interfacial tension between the air phase and the aqueous phase. Without wishing to be bound by theory, the Cellulose-Containing Compositions of the present invention seem to provide the surfactant to reduce the interfacial tension in these compositions where air bubbles and foams are being stabilized. This property may be beneficial in leavened and leavenable food products by stabilizing the air bubbles in the food product (such as breads, muffins, etc.) as disclosed herein, and in stabilizing the air bubble foams in ice cream as disclosed herein. Such air bubble stabilizing properties could also be of utility in other food products such as marshmallows, whipped cream, meringue, and the like, as well as food products that incorporate the foregoing or are “whipped” in order to create a lighter texture and/or different mouth-feel (e.g., mousse, whipped jello or pudding desserts and yogurts), and in other non-food products such as personal care products such as hair care, lotion, soap and make-up “mousse” or “whipped” products.

Another use for the Cellulose-Containing Compositions disclosed herein in the context of food products is to provide a lower calorie content food. A hazelnut spread (or peanut butter, or nut butter generally) may be provided at a similar viscosity, texture and taste, but with a lower caloric intake if made with the Cellulose-Containing Compositions described herein. Nut butters are generally high in calories (high fat/oil content), although relatively healthy in the context of a high calorie food. The Cellulose-Containing Compositions disclosed herein can support oil/water emulsions, and therefore allow the addition of water into the recipe. Despite an overall dilution due to addition of water, the texture, mouth-feel and taste can be maintained since the compositions additionally have a thickening effect and support spatial structure within the mixture. Further, doughs comprising the Cellulose-Containing Compositions that are fried in oils and fats (e.g., deep frying doughnuts) have a lower calorie content in comparison to similar products without these compositions cooked in a similar fashion. Accordingly, it is possible to both reduce the caloric intake (substituting water for nut butter content including fats/oils), while at the same time reducing cost (for the same reason).

### Personal Care and Cosmetic Products

Additionally, in various aspects, the Cellulose-Containing Compositions of the present invention, can be used, either alone or in combination with other components (e.g., in the form of the various other types of compositions described herein, such as a thickened composition, suspension, emulsion, solution, solid sticks, pressed powders, loose powders, gels, gel-cream, pastes, and the like), as a personal care or cosmetic product. The particles of the Cellulose-Containing Compositions themselves can provide an exfoliating, softening, or other desirable property (such as a feeling of rejuvenation) to a composition containing them (e.g., as a suspension of the particles in water, or as a suspension comprising the particles) when such a composition is applied to the skin (e.g., face, hands, feet, arms, legs, or any other skin on the body of a human or animal). In embodiments where the particles of the Cellulose-Containing Compositions of the present invention further comprise lignin, or where lignin is present in the compositions containing the particles comprising the compositions, the lignin also may provide (in conjunction with the cellulose particles) an exfoliating, softening, or other desirable property (such as a feeling of rejuvenation) to compositions containing such components.

Some compositions may be a combination of those described herein. In particular, a composition may technically be an emulsion, but it may also have suspended particles (e.g., particles other than the particles of the Cellulose-Containing Compositions described herein). Similarly, a thickened composition may also be an emulsion and/or a suspension. As one of ordinary skill in the art would understand, the Cellulose-Containing Compositions described herein need not be used only in compositions that are strictly considered to be emulsions, suspensions, or thickened compositions, etc., but rather the compositions of the present invention can be used in compositions that may have characteristics of any combination of these types of compositions (e.g., the composition may overlap to be considered a member of more than one category of composition type).

An example of such compositions, Cellulose-Containing Compositions of the present invention may function in one or more capacities as described above, is a personal care formulation (e.g., a beauty formulation, cosmetic formulation, a skin care formulation, etc.). The Cellulose-Containing Compositions of the present invention, may function, for example, as exfoliants, emulsifiers, texturizers, or both. The Cellulose-Containing Compositions, when functioning as exfoliants, texturizers, or both, may be present in a composition that is an emulsion, a suspension, a thickened composition, (or any other composition or type of composition described herein), or any combination thereof. In some embodiments, the Cellulose-Containing Compositions may be used directly (e.g., alone or minimally formulated), or used in a formulation comprising other ingredients (i.e., contain more ingredients than a minimally formulated composition), as an exfoliant. The Cellulose-Containing Compositions may also function (e.g., alone, minimally formulated, or more than minimally formulated) as texturizers. Accordingly, alone, minimally formulated, or more than minimally formulated, the Cellulose-Containing Compositions may function both as an exfoliant and as a texturizer. In such a capacity, the Cellulose-Containing Compositions may be present at any use level, including 100% of the solids (weight of solid particles based on total formulation solids). For example, in personal care formulations (e.g., beauty formulations, cosmetic formulations, skin care formulations, and so on), such as a facial scrub formulation, body scrub formulation, or lotion, the Cellulose-Containing Compositions described herein are present in the personal care formulation (weight % of dry solids of the Cellulose-Containing Compositions based on the total weight of the formulation) in an amount of from 0.05% to 50%. As used in this context, “minimally formulated” means that the formulation contains the Cellulose-Containing Compositions and a minimal amount of other ingredients that are necessary to produce a formulation satisfactory for its intended use (e.g., as a body scrub, a facial scrub, a skin lotion, etc.).

Personal care formulations may comprise additional ingredients in order to provide a different balance of properties. For example, personal care formulations may comprise one or more additional thickeners in order to optimize a formulation's viscosity or texture. In such cases, the Cellulose-Containing Compositions of the present invention may function as synergistic thickeners by providing a synergistic effect over and above that of the sum of the effect of the thickener(s) and the Cellulose-Containing Compositions. In such formulations, the Cellulose-Containing Compositions of the present invention may be present at levels described elsewhere herein, and the one or more additional thickener may be present at the levels normally associated with the specific thickener (suitable thickeners are described elsewhere herein and may be used for personal care formulations). For example, xanthan gum can be present in an amount of (weight % based on the total weight of the formulation) from 0.05% to 5%. Examples of other thickeners used in personal care products include, but are not limited to, polyacrylates, polyacrylamides, and ammonium acryloyldimethyltaurate/vinyl pyrrolidone copolymer.

Personal care formulations (e.g., beauty formulations, cosmetic formulations, body scrub formulations, facial scrub formulations, skin care formulations, hair care formulations, etc.) may additionally comprise other ingredients, including, for example, moisturizers, humectants, which can attract and bind water to improve hydration at the skin or hair surface, occlusive agents (such as oils), which may prevent or retard water leaving the skin or hair surface, emollients, which may create a smooth skin look or feel by filling in cracks or spaces in the skin, chemical peel agents (such as, e.g., glycolic acid, alpha hydroxyl acid, or salicylic acid). These acids are known to break down keratin, exfoliating the stratum corneum, promoting faster keratinization cycles, unclogging pores, leading to a brighter and radiant skin. These may help promote the growth of new skin cells for a rejuvenated skin, chemically exfoliate the skin and unclog pores, and inhibit growth of bacteria that causes acne), antioxidants, and a formulation biocide (e.g., one or more of phenoxyethanol, or a food-grade biocide such as a sodium benzoate/potassium sorbate/glycerin combination). Notably, some ingredients can function in one or more of the roles described above (e.g., an ingredient may be both an occlusive agent and an emollient), and therefore an attempt to categorize the ingredients should not be construed as limiting any ingredient to a particular function; such categorization is made merely to aid an ordinarily skilled person to understand the disclosure. Oils can be very effective for certain functions or purposes, but in some circumstances may not be desirable in skin care or hair care formulations, including body scrubs and facial scrubs, because the target formulation is intended to be water-based or “oil free,” or the formulations may require additional emulsifiers in order to fully disperse the oil(s) in aqueous formulations. The emulsifiers in some circumstances may impart a greasy feel to the formulation, which may be undesirable for some end uses. Moreover, some view formulation emulsifiers as potential skin irritants (one school of thought posits that the formulation emulsifiers aid in removal of natural lipids in the skin and hair when rinsed with water). Examples of emulsifiers that typically have been used in personal care formulations include fatty acids, such as stearic acid and palmitic acid, fatty alcohols such as stearyl alcohol and cetyl alcohol, fatty acid esters such as glyceryl monostearate and glycol mono- or di-stearate, as well as sodium stearyl glutamate, potassium cetyl phosphate and hydrogenated palm glycerides. Such species may be present in traditional personal care formulations at levels as high as 5-10% (weight % based on the total weight of the formulation). The Cellulose-Containing Compositions described herein can act as emulsifiers for oils, which renders the addition of emulsifiers to be unnecessary or undesirable.

In addition to convenience and cost advantages, the removal of the emulsifiers may also provide a lighter, less greasy feel to the composition, and also the emulsifier-free formulation may be milder to the skin. In any event, the above-described other ingredient(s), either alone or in any combination, can be present in the personal care formulation in an amount of (weight % of the other ingredient(s), individually or in combination, based on the total weight of the formulation) from 0.05% to 80%.

The personal care formulations can include suitable occlusive agents, humectants, emollients, or any combination thereof. Suitable occlusive agents include, for example, petrolatum, lanolin, mineral oil, silicones, dimethicone, argan oil, caprylic capric triglyceride, squalane, coconut oil, shea butter, or any combination thereof. Suitable humectants include, for example, propylene glycol, trehalose, glycerin, urea, hyaluronates, hyaluronic acid, pyrrolidonecarboxylate, or any combination thereof. Suitable emollients include, for example, fatty acid esters (octanoates such as octyl octanoate; laurates such as isoamyl laurate; adipates such as diisopropyl adipate; palmitates such as ethylhexyl palmitate), an oil, squalane, sesame oil, argan oil, algae extract, grape seed oil, caprylic capric triglyceride, cetyl stearate, glycerin, 1,3-butylene glycol, or any combination thereof. As noted earlier, some ingredients can function in one or more of the roles described above (e.g., an ingredient may be both an occlusive agent and an emollient). As used in the context of the other ingredients listed above, the term “moisturizer” is a category that includes an emollient, an occlusive agent, a humectant, or any combination thereof.

Suitable antioxidants for the personal care formulations include any of those known in the art, such as ascorbic acid. The Cellulose-Containing Compositions of the present invention may comprise lignin as described elsewhere herein. This lignin may also function as an antioxidant, which may render the addition of a separate antioxidant unnecessary, or it may work synergistically with an added antioxidant.

Personal care formulations, e.g., scrub formulations or skin care formulations, may additionally comprise other exfoliant materials. The Cellulose-Containing Compositions of the present invention provide a fine abrasive exfoliant (also known as a polish) because the particles thereof are small. It may be advantageous to additionally include an additional exfoliant, such as, one that has a similar D₅₀ particle size, a smaller D₅₀ particle size, a larger D₅₀ particle size, or any combination thereof. A suitable additional exfoliant can be a microbead (e.g., polymeric), a nanobead, microcrystalline cellulose, nanocellulose; crushed sea shells, nut shells, and other hard organic matter; appropriately sized table sugar or salt crystals; or any known exfoliant. The additional exfoliant can be present in an amount of (weight % based on the total weight of the formulation) 0.05% to 5%.

The Cellulose-Containing Compositions disclosed herein can find utility in many personal care products, for example, health and beauty or cosmetic products, such as, for example, a lotion, cream, ointment, serum, shampoo, conditioner, hair moisturizer, hairspray, hair gel, deodorant, facial or body wash, facial or body scrub, cleansers, exfoliant, emollient, moisturizer, soap, foundation make up, BB cream, CC cream, eye cream, sunscreen, anti-acne serum or cream or lotion, cellular serum or cream or lotion, facial or body mask, blush, eyeshadow, mascara, lipstick or lip balm, or clay, kaolin or mud suspension. Depending on the product type, the use of the Cellulose-Containing Compositions disclosed herein in a personal care product may present one or more of the following non-limiting beneficial properties: ability to remove undesirable chemicals such as emulsifiers from the formulation, resulting in a milder product (less irritant chemicals); a less oily or greasy feel to the product (described as a “lighter” or “airy” composition) resulting from removal of emulsifiers and better incorporation of oils and other occlusive agents; reduced quantities of viscosifying agents, since the compositions act as a thickener, and additionally may show a synergistic thickening effect with other thickeners; a mattifying and/or line filling effect for the skin, since the small particle size facilitates access to and filling of uneven skin and wrinkles; and a rejuvenating sensation for the skin, since the small particles act as a non-irritating exfoliant. Moreover, the compositions of the present invention may facilitate the delivery of either suspended solid- or immiscible liquid-active ingredients to the skin, since the cellulose-containing compositions are able to stabilize solids in suspension and emulsions comprised of immiscible liquids.

### Aspects of the Present Invention

Accordingly, in a first aspect of the present invention, a method for the preparation of a cellulose-containing composition is provided, comprising the steps of:
(a) forming a first slurry comprising a cellulose-containing feedstock, water, and acid;
(b) subjecting the first slurry to rapid heating to a first temperature, by addition of subcritical, near critical or supercritical water, at a first pressure, thereby forming a second slurry;
(c) maintaining the second slurry at the first temperature and first pressure for a first time period;
(d) rapidly depressurizing the second slurry to form a third slurry at a second temperature and a second pressure; and
(e) separating the liquid portion from the solid portion of the third slurry, the solid portion of which comprises the cellulose-containing composition;
wherein the first temperature is in the range of 180° C. to 280° C.; and
wherein the cellulose contained in the cellulose-containing composition has a weight average molecular weight between 40,000 grams/mole and 155,000 grams/mole.

In a second aspect of the present invention, the method according to the first aspect is provided, wherein the cellulose contained in the cellulose-containing composition has a weight average molecular weight between 75,000 grams/mole and 155,000 grams/mole.

In a third aspect of the present invention, the method according to either of the first or second aspects is provided, wherein the cellulose-containing feedstock comprises cellulose, hemicellulose, or lignin, or a mixture thereof.

In a fourth aspect of the present invention, the method according to any one of the first, second or third aspects is provided, wherein the cellulose-containing feedstock is selected from woody biomass, pea hulls, corn fiber, soy hulls, citrus residues, cocoa shells, cotton, oat fiber, bran fiber, bagasse, crop grasses, hemp, cereals, fruits, vegetables, paper products and wood products, and mixtures of two or more thereof.

In a fifth aspect of the present invention, the method according to any of the first through fourth aspects is provided, wherein the first temperature is in the range from 200° C. to less than or equal to 260° C.

In a sixth aspect of the present invention, the method according to any of the first through fifth aspects is provided, wherein the first time period is from 0.5 second to 4 seconds.

In a seventh aspect of the present invention, the method according to any one of the first through sixth aspects is provided, wherein the rapidly depressurizing occurs over a period of less than 5% of the first time period.

In an eighth aspect of the present invention, the method according to any one of the first through seventh aspects is provided, wherein the rapid heating occurs over a period of from 5% to 50% of the first time period.

In a ninth aspect of the present invention, the method according to any one of the first through eighth aspects is provided, wherein the first pressure is in the range from 1,500 psi to 3,600 psi.

In a tenth aspect of the present invention, the method according to any one of the first through ninth aspects is provided, wherein the second pressure is in the range of 5 psi to 75 psi.

In an eleventh aspect of the present invention, the method according to any one of the first through tenth aspects is provided, wherein the second temperature is in the range from 45° C. to 150° C.

In a twelfth aspect of the present invention, the method according to any one of the first through eleventh aspects is provided, wherein the acid is an inorganic acid, or an organic acid, or a mixture thereof.

In a thirteenth aspect of the present invention, the method according to the twelfth aspect is provided, wherein the inorganic acid is selected from sulfuric acid, sulfonic acid, phosphoric acid, phosphonic acid, nitric acid, nitrous acid, hydrochloric acid, hydrofluoric acid, hydrobromic acid, and hydroiodic acid, and mixtures thereof.

In a fourteenth aspect of the present invention, the method according to the thirteenth aspect is provided, wherein the acid is selected from sulfuric acid, nitric acid and hydrochloric acid, and mixtures thereof.

In a fifteenth aspect of the present invention, the method according to the thirteenth aspect is provided, wherein the organic acid is selected from an aliphatic carboxylic acid, an aromatic carboxylic acid, a dicarboxylic acid, an aliphatic fatty acid, an aromatic fatty acid, an amino acid, an alkyl sulfonic acid, and an acyl sulfonic acid, and mixtures thereof.

In a sixteenth aspect of the present invention, the method according to the fifteenth aspect is provided, wherein the aliphatic carboxylic acid is formic acid or acetic acid, or a mixture thereof.

In a seventeenth aspect of the present invention, the method according to the fifteenth aspect is provided, wherein the aromatic carboxylic acid is selected from benzoic acid and salicylic acid, and mixtures thereof.

In an eighteenth aspect of the present invention, the method according to the fifteenth aspect is provided, wherein the dicarboxylic acid is selected from oxalic acid, phthalic acid, sebacic acid and adipic acid, and mixtures thereof.

In a nineteenth aspect of the present invention, the method according to the fifteenth aspect is provided, wherein the aliphatic fatty acid is selected from oleic acid, palmitic acid and stearic acid, and mixtures thereof.

In a twentieth aspect of the present invention, the method according to the fifteenth aspect is provided, wherein the aromatic fatty acid is phenylstearic acid.

In a twenty-first aspect of the present invention, the method according to the fifteenth aspect is provided, wherein the alkyl sulfonic acid is methane sulfonic acid.

In a twenty-second aspect of the present invention, the method according to any one of the first through twenty-first aspects is provided, wherein the pH of the first slurry is in the range from pH 1.0 to pH 3.0.

In a twenty-third aspect of the present invention, the method according to any one of the first through twenty-second aspects is provided, wherein the cellulose-containing composition comprises from 50% to 100% of cellulose based on the total weight of the cellulose-containing composition.

In a twenty-fourth aspect of the present invention, the method according to any one of first through twenty-third aspects is provided, wherein the cellulose-containing composition comprises from 60% to 90% cellulose based on the total weight of the cellulose-containing composition.

In a twenty-fifth aspect of the present invention, the method according to any one of the first through twenty-fourth aspects is provided, wherein the cellulose-containing composition comprises microcrystalline cellulose.

In a twenty-sixth aspect of the present invention the method according to any of the first through twenty-fifth aspects is provided, wherein the cellulose-containing composition comprises at least 50% microcrystalline cellulose based on the total weight of the cellulose-containing composition.

In a twenty-seventh aspect of the present invention, the method according to any one of the first through twenty-sixth aspects is provided, wherein the cellulose containing composition comprises a range of between 50% microcrystalline cellulose to 90% microcrystalline cellulose based on the total weight of the cellulose-containing composition.

In a twentieth-eighth aspect of the present invention, the method according to any one of the first through aspects is provided, wherein the cellulose-containing composition, comprises at least 75% microcrystalline cellulose based on the total weight of the cellulose-containing composition.

In a twenty-ninth aspect of the present invention, the method according to any one of the first through twenty-eighth aspects is provided, wherein the cellulose-containing composition comprises a range of microcrystalline cellulose of from 80% to 100% microcrystalline cellulose based on the weight of the cellulose in the cellulose-containing composition.

In a thirtieth aspect of the present invention, the method according to any one of the first through twenty-ninth aspect is provided, wherein the cellulose-containing composition comprises a range of microcrystalline cellulose of from 90% to 100% microcrystalline cellulose based on the weight of the cellulose in the cellulose-containing composition.

In a thirty-first aspect of the present invention, the method according to any one of the first through thirtieth aspects is provided, wherein the cellulose-containing composition further comprises lignin or hemicellulose, or a mixture thereof.

In a thirty-second aspect of the present invention, the method according to any one of the twenty-fifth through thirty-first aspects is provided, wherein the particles of the microcrystalline cellulose have an aspect ratio of between 2.0 and 10.0.

In a thirty-third aspect of the present invention, the method according to any one of the twenty-fifth through thirty-second aspects is provided, wherein the particles of the microcrystalline cellulose have an aspect ratio of between 2.5 and 8.5.

In a thirty-fourth aspect of the present invention, the method according to any one of the first through thirty-third aspects is provided, wherein the particles of the cellulose-containing composition have a mean particle size between 20 μm and 110 μm.

In a thirty-fifth aspect of the present invention, the method according to any one of the first through thirty-fourth aspects is provided, wherein the separating is accomplished using gravity separation, centrifugal separation, centripetal separation, filtration, or a combination thereof.

In a thirty-sixth aspect of the present invention, the cellulose-containing composition prepared by the method according to any one first through thirty-fifth aspects is provided.

In a thirty-seventh aspect of the present invention, a cellulose-containing composition according to either the thirty-sixth aspect, or the one hundred and twenty-second aspect is provided, comprising from 50% to 100% of cellulose based on the total dry weight of the cellulose-containing composition.

In a thirty-eighth aspect of the present invention, the cellulose-containing composition according to the thirty-seventh aspect is provided, comprising from 60% to 90% of cellulose based on the total dry weight of the cellulose-containing composition.

In a thirty-ninth aspect of the present invention, a cellulose-containing composition according to either of the thirty-seventh or thirty-eighth aspects is provide, wherein at least a portion of the cellulose-containing composition comprises microcrystalline cellulose.

In a fortieth aspect of the present invention, a cellulose-containing composition according to the thirty-ninth aspect is provided, comprising at least 50% microcrystalline cellulose based on the total dry weight of the cellulose-containing composition.

In a forty-first aspect of the present invention, a cellulose-containing composition according to the fortieth aspect is provided, comprising a range of between 50% microcrystalline cellulose to 90% microcrystalline cellulose based on the total dry weight of the cellulose-containing composition.

In a forty-second aspect of the present invention, a cellulose-containing composition according to the fortieth aspect is provided, comprising at least 75% microcrystalline cellulose based on the weight of the cellulose in the cellulose-containing composition.

In a forty-third aspect of the present invention, a cellulose-containing composition according to the forty-second aspect is provided, comprising a range of microcrystalline cellulose of from 80% to 100% microcrystalline cellulose based on the weight of the cellulose in the cellulose-containing composition.

In a forty-fourth aspect of the present invention, a cellulose-containing composition according to the forty-third aspect is provided, comprising a range of microcrystalline cellulose of from 90% to 100% microcrystalline cellulose based on the weight of the cellulose in the cellulose-containing composition.

In a forty-fifth aspect of the present invention, the cellulose-containing composition according to any one of the thirty-sixth through forty-fourth aspects is provided, wherein the cellulose-containing composition further comprises lignin, hemicellulose, or a mixture thereof.

In a forty-sixth aspect of the present invention, the cellulose-containing composition according to any one of the thirty-sixth through forty-fifth aspects is provided, wherein the particles of the microcrystalline cellulose have an aspect ratio of between about 2.0 and 10.0.

In a forty-seventh aspect of the present invention, the cellulose-containing composition according to any one of the thirty-sixth through forty-sixth aspects is provided, wherein the particles of the microcrystalline cellulose have an aspect ratio of between 2.5 and 8.5.

In a forty-eighth aspects of the present invention, the cellulose-containing composition according to any one of the thirty-sixth through forty-seventh aspects is provided, wherein the cellulose contained in the cellulose-containing composition has a weight average molecular weight between 40,000 grams/mole and 155,000 grams/mole.

In a forty-ninth aspect of the present invention, the cellulose-containing composition according to any one of the thirty-sixth through forty-eighth aspects is provided, wherein the cellulose contained in the cellulose-containing composition has a weight average molecular weight between 75,000 grams/mole and 125,000 grams/mole.

In a fiftieth aspect of the present invention, the cellulose-containing composition according to any one of the thirty-sixth through forty-ninth aspects is provided, wherein the particles of the cellulose-containing composition have a mean particle size between 20 μm and 110 μm.

In a fifty-first aspect of the present invention, a baked good with enriched fiber, comprising the composition according to any one of the thirty-sixth through fiftieth aspects.

In a fifty-second aspect of the present invention, an egg replacement for baked goods, comprising the composition according to any one of the thirty-sixth through fiftieth aspects is provided.

In a fifty-third aspect of the present invention, an oil in water emulsion stabilizer, comprising the composition according to any one of the thirty-sixth through fiftieth aspects is provided.

In a fifty-fourth aspect of the present invention, a texturizer is provided, comprising the composition according to any one of the thirty-sixth through fiftieth aspects.

In a fifty-fifth aspect of the present invention, a rheology modifier that increases the viscosity of an aqueous system is provided, comprising the composition according to any one of the thirty-sixth through fiftieth aspects

In a fifty-sixth aspect of the present invention, a cosmetic comprising an emulsion stabilizer is provided, comprising the composition according to one of the thirtieth through fiftieth aspects.

In a fifty-seventh aspect of the present invention, a cosmetic comprising a thickener is provided, comprising the composition according to any one of the thirtieth through fiftieth aspects.

In a fifty-eighth aspect of the present invention, a cosmetic comprising a texturizer is provided, comprising the composition according to any one of the thirtieth through fiftieth aspects.

In a fiftieth aspect of the present invention, a cosmetic comprising a bulking agent is provided, comprising the composition according to any one of the thirty-sixth through fiftieth aspects.

In a sixtieth aspect of the present invention, a cosmetic comprising a filler is provided, comprising the composition according to any one of the thirty-sixth through fiftieth aspects.

In a sixty-first aspect of the present invention, a paint comprising the composition according to any one of the thirtieth sixth through fiftieth aspects is provided.

In a sixty-second aspect of the present invention, a coating comprising a rheology modifier is provided, comprising the composition according to any one of the thirty-sixth through fiftieth aspects.

In a sixty-third aspect of the present invention, a carrier for a drug delivery system is provided, comprising the composition according to any one of the thirty-sixth through fiftieth aspects.

In a sixty-fourth aspect of the present invention, a pharmaceutical excipient is provided, comprising the composition according to any one of the thirty-sixth through fiftieth aspects.

In a sixty-fifth aspect of the present invention, a nutraceutical excipient is provided, comprising the composition according to any one of the thirty-sixth through fiftieth aspects.

In a sixty-sixth aspect of the present invention, a method for the preparation of a cellulose-containing composition is provided, comprising the steps of:
(a) forming a first slurry comprising a cellulose-containing feedstock, water, and acid;
(b) subjecting the first slurry to rapid heating to a first temperature, by addition of subcritical, near critical or supercritical water, at a first pressure, thereby forming a second slurry;
(c) maintaining the second slurry at the first temperature and first pressure for a first time period; and
(d) rapidly depressurizing the second slurry to form a third slurry at a second temperature and a second pressure;
(e) concentrating the third slurry to form the cellulose-containing composition;
wherein the first temperature is in the range of 180° C. to 280° C.; and
   wherein the cellulose contained in the cellulose-containing composition has a weight average molecular weight between 40,000 grams/mole and 155,000 grams/mole.

In a sixty-seventh aspect of the present invention, the method according to the sixty-sixth aspect is provided, wherein the concentrating is performed by filtration of the third slurry to form a residue and a filtrate, concentrating the filtrate of the third slurry, and adding the concentrated filtrate back into the residue.

In a sixty-eighth aspect of the present invention, the method according to the sixty-sixth aspect is provided, wherein the concentrating of the third slurry is performed by centrifugation, to form a pellet and a supernatant, the supernatant is concentrated, and then added back to the pellet.

In a sixty-ninth aspect of the present invention, the method according to the sixty-sixth aspect is provided, wherein the concentrating is performed by concentration of the third slurry in vacuo.

In a seventieth aspect of the present invention, the method according to any one of the sixty-sixth through sixty-ninth aspects is provided, wherein the cellulose contained in the cellulose-containing composition has a weight average molecular weight between 75,000 grams/mole and 155,000 grams/mole.

In a seventy-first aspect of the present invention, the method according to any one of the sixty-sixth through seventieth aspects is provided, wherein the cellulose-containing feedstock comprises cellulose, hemicellulose, or lignin, or a mixture thereof.

In a seventy-second aspect of the present invention, the method according to any one of the sixty-ninth through seventy-first aspects is provided, wherein the cellulose-containing feedstock is selected from wood products, pea hulls, corn fiber, soy hulls, citrus residues, cocoa shells, cotton, oat fiber, bran fiber, bagasse, crop grasses, hemp, cereals, fruits, vegetables, paper products and wood products, and mixtures thereof.

In a seventy-third aspect of the present invention, the method according to any one of the sixty-sixth through seventy-second aspects is provided, wherein the first temperature is in the range from 200° C. to less than or equal to 260° C.

In a seventy-fourth aspect of the present invention, the method according to any of sixty-sixth through seventy-third aspects is provided, wherein the first time period is from 0.5 seconds to 4 seconds.

In a seventy-fifth aspect of the present invention, the method according to any one of sixty-sixth through seventy-fourth aspects is provided, wherein the rapidly depressurizing occurs over a period of less than 5% of the first time period.

In a seventy-sixth aspect of the present invention, the method according to any one of the sixty-sixth through seventy-fifth aspects is provided, wherein the rapid heating occurs over a period of from 5% to 50% of the first time period.

In a seventy-seventh aspect of the present invention, the method according to any one of the sixty-sixth through seventy-sixth aspects is provided, wherein the first pressure is in the range from 1,500 psi to 3,600 psi.

In a seventy-eighth aspect of the present invention, the method according to any one of the sixty-sixth through seventy-seventh aspects is provided, wherein the second pressure is in the range of 5 psi to 75 psi.

In a seventy-ninth aspect of the present invention, the method according to any one of the sixty-sixth through seventy-eighth aspects is provided, wherein the second temperature is in the range from 45° C. to 150° C.

In an eightieth aspect of the present invention, the method according to any one of the sixty-sixth through seventy-eighth aspects is provided, wherein the acid is an inorganic acid, or an organic acid, or a mixture thereof.

In an eighty-first aspect of the present invention, the method according to the eightieth aspect is provided, wherein the inorganic acid is selected from sulfuric acid, sulfonic acid, phosphoric acid, phosphonic acid, nitric acid, nitrous acid, hydrochloric acid, hydrofluoric acid, hydrobromic acid, and hydroiodic acid, and mixtures thereof.

In an eighty-second aspect of the present invention, the method according to the eighty-first aspect is provided, wherein the acid is selected from sulfuric acid, nitric acid and hydrochloric acid, and mixtures thereof.

In an eighty-third aspect of the present invention, the method according to the eightieth aspect is provided, wherein the organic acid is selected from an aliphatic carboxylic acid, an aromatic carboxylic acid, a dicarboxylic acid, an aliphatic fatty acid, an aromatic fatty acid, an amino acid, an alkyl sulfonic acid, and an acyl sulfonic acid, and mixtures thereof.

In an eighty-fourth aspect of the present invention, the method according to the eighty-third aspect is provided, wherein the aliphatic carboxylic acid is formic acid or acetic acid, or a mixture thereof.

In an eighty-fifth aspect of the present invention, the method according to the eighty-third aspect is provided, wherein the aromatic carboxylic acid is selected from benzoic acid and salicylic acid, and mixtures thereof.

In an eighty-sixth aspect of the present invention, the method according to the eighty-third aspect is provided, wherein the dicarboxylic acid is selected from oxalic acid, phthalic acid, sebacic acid and adipic acid, and mixtures thereof.

In an eighty-seventh aspect of the present invention, the method according to the eighty-third aspect is provided, wherein the aliphatic fatty acid is selected from oleic acid, palmitic acid and stearic acid, and mixtures thereof.

In an eighty-eighth aspect of the present invention, the method according to the eighty-third aspect is provided, wherein the aromatic fatty acid is phenylstearic acid.

In an eighty-ninth aspect of the present invention, the method according to the eighty-third aspect is provided, wherein the alkyl sulfonic acid is methane sulfonic acid.

In a ninetieth aspect of the present invention, the method according to any one of the sixty-sixth through eighty-ninth aspects is provided, wherein the pH of the first slurry is from pH 1.0 to pH 3.0.

In a ninety-first aspect of the present invention, A cellulose-containing composition prepared according to the method according to any one of the sixty-sixth through ninetieth aspects is provided.

In a ninety-second aspect of the present invention, a cellulose-containing composition according to the ninety-first aspect, or the one hundred and twenty-first aspect, or the one hundred and twenty-third aspect, is provided, comprising from 25% to 50% of cellulose based on the total dry weight of the cellulose-containing composition.

In a ninety-third aspect of the present invention, the cellulose-containing composition according to the ninety-second aspect is provided, comprising from 40% to 50% of cellulose based on the total dry weight of the cellulose-containing composition.

In a ninety-fourth aspect of the present invention, a cellulose-containing composition according to either of the ninety-second or ninety-third aspects is provided, wherein at least a portion of the cellulose-containing composition comprises microcrystalline cellulose.

In a ninety-fifth aspect of the present invention, a cellulose-containing composition according to the ninety-fourth aspect is provided, comprising at least 25% microcrystalline cellulose based on the total dry weight of the cellulose-containing composition.

In a ninety-sixth aspect of the present invention, a cellulose-containing composition according to the ninety-fifth aspect is provided, comprising a range of between 25% microcrystalline cellulose to 50% microcrystalline cellulose based on the total dry weight of the cellulose-containing composition.

In a ninety-seventh aspect of the present invention, a cellulose-containing composition according to the ninety-fifth aspect is provided, comprising at least 75% microcrystalline cellulose based on the weight of the cellulose in the cellulose-containing composition.

In a ninety-eighty aspect of the present invention, a cellulose-containing composition according to the ninety-seventh aspect is provided, comprising a range of microcrystalline cellulose of from 80% to 100% microcrystalline cellulose based on the weight of the cellulose in the cellulose-containing composition.

In a ninety-ninth aspect of the present invention, a cellulose-containing composition according to the ninety-seventh aspect is provided, comprising a range of microcrystalline cellulose of from 90% to 100% microcrystalline cellulose based on the weight of the cellulose in the cellulose-containing composition.

In a one-hundredth aspect of the present invention, the cellulose-containing composition according to any one of the ninety-first through ninety-ninth aspects is provided, wherein the cellulose-containing composition further comprises lignin, hemicellulose, or a mixture thereof.

In a one hundred and first aspect of the present invention, the cellulose-containing composition according to any one of the ninety-first through one hundredth aspects is provided, wherein the particles of the microcrystalline cellulose have an aspect ratio of between about 2.0 and 10.0.

In a one hundred and second aspect of the present invention, the cellulose-containing composition according to any one of the ninety-first through one hundred and first aspects is provided, wherein the particles of the microcrystalline cellulose have an aspect ratio of between 2.5 and 8.5.

In a one hundred and third aspect of the present invention, the cellulose-containing composition according to any one of the ninety-first through one hundred and second aspects is provided, wherein the cellulose contained in the cellulose-containing composition has a weight average molecular weight between 40,000 grams/mole and 155,000 grams/mole.

In a one hundred and fourth aspect of the present invention, the cellulose-containing composition according to any one of the ninety-first through one hundred and third aspects is provided, wherein the cellulose contained in the cellulose-containing composition has a weight average molecular weight between 75,000 grams/mole and 125,000 grams/mole.

In a one hundred and fifth aspect of the present invention, the cellulose-containing composition according to any one of the ninety-first through one hundred and fourth aspects is provided, wherein the particles of the cellulose-containing composition have a mean particle size between 20 μm and 110 μm.

In a one hundred and sixth aspect of the present invention, a baked good with enriched fiber is provided, comprising the composition according to any one of the ninety-first through one hundred and fifth aspects.

In a one hundred and seventh aspect of the present invention, an egg replacement for baked goods is provided, comprising the composition according to any one of the ninety-first through one hundred and fifth aspects.

In a one hundred and eighth aspect of the present invention, an oil in water emulsion stabilizer is provided, comprising the composition according to any one of the ninety-first through one hundred and fifth aspects.

In a one hundred and ninth aspect of the present invention, a texturizer is provided, comprising the composition according to any one of the ninety-first through one hundred and fifth aspects.

In a one hundred and tenth aspect of the present invention, a rheology modifier that increases the viscosity of an aqueous system is provided, comprising the composition any one of the ninety-first through one hundred and fifth aspects.

In a one hundred and eleventh aspect of the present invention, a cosmetic comprising an emulsion stabilizer is provided, comprising the composition according to any one of the ninety-first through one hundred and fifth aspects.

In a one hundred and twelfth aspect of the present invention, a cosmetic comprising a thickener is provided, comprising the composition according to any one of the ninety-first through one hundred and fifth aspects.

In a one hundred and thirteenth aspect of the present invention, a cosmetic comprising a texturizer is provided, comprising the composition according to any one of the ninety-first through one hundred and fifth aspects.

In a one hundred and fourteenth aspect of the present invention, a cosmetic comprising a bulking agent is provided, comprising the composition according to any one of the ninety-first through one hundred and fifth aspects.

In a one hundred and fifteenth aspect of the present invention, a cosmetic comprising a filler is provided, comprising the composition according to any one of the ninety-first through one hundred and fifth aspects.

In a one hundred and sixteenth aspect of the present invention, a paint comprising the composition according to any one of the ninety first through one hundred and fifth aspects is provided.

In a one hundred and seventeenth aspect of the present invention, a coating comprising a rheology modifier is provided, comprising the composition according to any one of the ninety-first through one hundred and fifth aspects.

In a one hundred and nineteenth aspect of the present invention, a carrier for a drug delivery system is provided, comprising the composition according to any one of the ninety-first through one hundred and fifth aspects.

In a one hundred and nineteenth aspect of the present invention, a pharmaceutical excipient is provided, comprising the composition according to any one of the ninety-first through one hundred and fifth aspects.

In a one hundred and twentieth aspect of the present invention, a nutraceutical excipient is provided, comprising the composition according to any one of the ninety first through one hundred and fifth aspects

In a one hundred and twenty-first aspect of the present invention a composition according to any one of the ninety-first through one hundred and fifth aspects is provided, comprising a soluble glucose monomer, a soluble glucose oligomer, a soluble xylose monomer, a soluble xylose oligomer, or a combination thereof.

In a one hundred and twenty-second aspect of the present invention, a composition according to any one of the thirty-sixth through fiftieth aspects is provided, comprising water, so as to provide a moisture content of from about 5 wt % to about 90 wt %.

In a one hundred and twenty-third aspect of the present invention, a composition according to any one of the ninety-first through one hundred and fifth aspects, or the one hundred and twenty-first aspect, is provided, comprising water, so as to provide a moisture content of from about 5 wt % to about 90 wt %.

### EXAMPLES

Determination of Molecular Weight of Cellulose: Solvent exchange was performed on the aqueous slurry samples (approximately 150 mg solid) as described in US 2019/0008749 A1, which is a modified method based on the article Dupont, Polymer, “Cellulose in lithium chloride/N,N-dimethylacetamide, optimization of a dissolution method using paper substrates and stability of the solutions,” Vol. 44, (2003), 4117-4126. Aqueous slurries were centrifuged, then the solids were re-suspended twice in methanol for 45 minutes each at room temperature while stirring (filter and re-suspend solids in fresh methanol after each activating), then the solids were re-suspended in N,N-dimethylacetamide (DMAc) overnight at room temperature with stirring (followed by filtration of solids), and finally the solids were diluted to 2 g by 8 wt % LiCl in DMAc and allowed to stir for three days. The samples are then diluted ten-fold to 0.8 wt % LiCl in DMAC prior to analyzing via GPC. 6 narrow pullulan standards (MWp range of 5,900-708,000 g/mol) were dissolved directly in 0.8 wt % LiCl in DMAc at a concentration of 5 mg pullulan/g solvent by heating at 55° C. and allowing to stir for 24 h. All samples and standards are filtered through 0.45 μm Nylon syringe filters into 1.5 mL glass vials prior to analysis.

GPC analysis was carried out on a Malvern Viscotek GPCmax instrument equipped with a triple detector array 305. OmniSEC 4.7 software was used for data analysis. Separation was achieved via one Viscotek LT5000L and two Viscotek T4000 columns connected in series using 0.8 wt % LiCl in DMAc as the eluent and employing the RID. The samples were analyzed with the following acquisition parameters: injection volume of 100 μL, flow rate of 1 mL/min, with column and detector temperatures set at 60° C. The 6 pullulan standards were used to construct a conventional calibration curve, from which the molar mass distribution data (MMD) was calculated.

Particle size distribution was determined using a Beckman LS 13 320 Laser Diffraction Particle Size Analyzer running LS 13 320 software.

Table 1 shows the particle size distribution of the particles in the cellulose-containing compositions as prepared in Examples 1 to 6.

TABLE 1

| Example | D₁₀ | D₂₅ | D₅₀ | D₇₅ | D₉₀ |
|---|---|---|---|---|---|
| Number | (μm) | (μm) | (μm) | (μm) | (μm) |
| 1 | 13.5 | 25.9 | 48.2 | 81.3 | 132 |
| 2 | 26.5 | 56.6 | 107 | 171 | 243 |
| 3 | 5.92 | 13.8 | 28.6 | 51.5 | 76.1 |
| 4 | 22.0 | 48.5 | 91.0 | 146 | 206 |
| 5 | 9.22 | 19.3 | 35.3 | 61.3 | 87.5 |
| 6 | 5.36 | 13.4 | 31.6 | 65.3 | 119 |

Aspect ratios were determined microscopically using an Olympus Model BX53F microscope with image analysis using Olympus cellSens software.

The percentage of microcrystalline cellulose was determined by X-Ray powder diffraction analysis using a Rigaku SmartLab X-ray Diffractometer XE system. The XRD system has a high-brilliance 9 kW Rotating Anode Cu X-Ray source. Data analysis was performed using SmartLab Studio II software from Rigaku.

Table 2 shows the percentage of cellulose, based on the weight the suspended solids, and the percentage of microcrystalline cellulose based on the total weight of cellulose (% MCC) in the cellulose-containing compositions as prepared in Examples 1 to 6

TABLE 2

| Example Number | % Cellulose | % MCC |
|---|---|---|
| 1 | 68 | 100 |
| 2 | 61 | ND |
| 3 | 87 | ND |
| 4 | 65 | ND |
| 5 | 79 | 100 |
| 6 | 54 | ND |

(ND, Not Determined)

Compositional analysis of samples is performed on a dry basis. Samples are dried in an oven at 105° C. to 110° C. for at least 2 hours to remove water. The dried sample is then ground with a mortar and pestle to break apart large agglomerates. For ash content, the dry solids are heated in a furnace at 575° C. for 10 hours. The remaining mass following this treatment is characterized as ash.

For acid insoluble lignin and carbohydrate analysis, the dry solids are subjected to a strong acid digestion. The dry solids are combined with 72 wt % sulfuric acid in a pressure tube and heated at 30° C. for 1 hour in a sonicating water bath. After this time, the sample is allowed to cool and then vacuum filtered. The remaining solids are identified as acid-insoluble lignin. The filtrate containing the carbohydrate oligomers, monomers, and associated degradation products are analyzed via HPLC. Carbohydrate content of the slurry liquor (i.e., the dissolved solids of the products of the processes of the present application) is also analyzed via the same acid hydrolysis method, followed by HPLC analysis. The slurry liquor is also analyzed by HPLC via direct injection without acid hydrolysis.

Table 3 shows the results of these determinations for Examples 1 to 6, wherein % XMGFA indicates the percentage, on a dried basis of combined xylose, mannose, galactose, fructose and arabinose. Mass balance includes % suspended solids and % lignin, as disclosed in the examples.

TABLE 3

| | % Glucose | % Glucose | % Hemi- | % XMGFA | % XMGFA | | Mass |
|---|---|---|---|---|---|---|---|
| Example | Monomer | Oligomers | cellulose | Monomers | Oligomers | % Ash | Balance |
| 1 | 2.52 | 7.75 | 7.51 | 0.00 | 12.54 | 3.77 | 87.22 |
| 2 | 0.00 | 10.93 | 12.43 | 0.00 | 8.69 | 3.41 | 91.60 |
| 3 | 2.90 | 11.99 | 2.29 | 2.22 | 13.98 | 2.90 | 95.95 |
| 4 | 0.00 | 9.97 | 9.51 | 0.59 | 10.17 | 3.13 | 87.93 |
| 5 | 7.24 | 9.02 | 4.32 | 0.07 | 6.67 | 3.10 | 90.86 |
| 6 | 1.75 | 6.10 | 2.17 | 0.15 | 12.11 | 4.67 | 103.76 |

### Example 1

A slurry of about 11% concentration of pea hulls in water, and sufficient sulfuric acid to provide a pH of 1.8 were mixed at the reactor inlet with water heated to a sub-critical temperature. The combination of both streams produced a reactor inlet temperature targeted at 213° C. The reactor pressure was controlled by a let-down valve at a targeted pressure of 2530 psi. The reaction time (time that a particle of cellulose-containing feedstock spent in the reactor) was 1.1 seconds. The product from this example yielded 66% suspended solids (based on the dry weight of the cellulose-containing feedstock). A portion of the slurry of this example, prior to separation of the suspended solids from the liquid, was concentrated and homogenized with a high shear mixer, to provide the starting material for Examples 8 and 9, and to provide a sample for the analyses shown in Table 3. The aspect ratio of the microcrystalline cellulose particles of this example was in the range of 4.11 to 8.02. The weight average molecular weight of the cellulose prepared in this example was determined to be 150,951 grams per mole. The lignin content of this example was determined to be 7.9%.

### Example 2

A slurry of 10.7% concentration of pea hulls in water, and sufficient sulfuric acid to provide a pH of 1.8 were mixed at the reactor inlet with water heated to a sub-critical temperature. The combination of both streams produced a reactor inlet temperature targeted at 180° C. The reactor pressure was controlled by a let-down valve at a pressure of 2490 psi. The reaction time (time that a particle of the cellulose-containing feedstock spent in the reactor, on average) was 1.1 seconds. The product from this example yielded 79% suspended solids (based on the dry weight of the suspended solids). A portion of the slurry of this example, prior to separation of the suspended solids from the liquid, provided a sample for the analyses shown in Table 3. The aspect ratio of the microcrystalline cellulose particles of this example was in the range of 2.62 to 6.84. The weight average molecular weight of the cellulose prepared in this example was determined to be 125,334 grams per mole. The lignin content of this example was determined to be 8.3%.

### Example 3

A slurry of 10.7% concentration of pea hulls in water, and sufficient sulfuric acid to provide a pH of 1.8 were mixed at the reactor inlet with water heated to a sub-critical temperature. The combination of both streams produced a reactor inlet temperature targeted at 248° C. The reactor pressure was controlled by a let-down valve at a pressure of 2540 psi. The reaction time (time that a particle of the cellulose-containing feedstock spent in the reactor, on average) was 1.1 seconds. The product from this example yielded 54% suspended solids. A portion of the slurry of this example, prior to separation of the suspended solids from the liquid, provided a sample for the analyses shown in Table 3. The aspect ratio of the microcrystalline cellulose particles of this example was in the range of 3.40 to 7.51. The weight average molecular weight of the cellulose prepared in this example was determined to be 44,891 grams per mole. The lignin content of this example was determined to be 12.6%.

### Example 4

A slurry of 11.1% concentration of pea hulls in water, and sufficient sulfuric acid to provide a pH of 2.7 were mixed at the reactor inlet with water heated to a sub-critical temperature. The combination of both streams produced a reactor inlet temperature targeted at 208° C. The reactor pressure was controlled by a let-down valve at a pressure of 2510 psi. The reaction time (time that a particle of cellulose-containing feedstock spent in the reactor, on average) was 1.1 seconds. The product from this example yielded 75% suspended solids. A portion of the slurry of this example, prior to separation of the suspended solids from the liquid provided a sample for the analyses shown in Table 3. The aspect ratio of the microcrystalline cellulose particles of this example was in the range of 2.59 to 5.61. The weight average molecular weight of the cellulose prepared in this example was determined to be 84,749 grams per mole. The lignin content of this example was determined to be 5.6%.

### Example 5

A slurry of 10.4% concentration of pea hulls in water, and sufficient sulfuric acid to produce a pH of 1.3 were mixed with water heated to a sub-critical temperature. The combination of both streams produced a reactor inlet temperature targeted at 209° C. The reactor pressure was controlled by a let-down valve at a pressure of 2490 psi. The reaction time (time that a particle of cellulose-containing feedstock spent in the reactor, on average) was 1.1 seconds. The product from this example yielded 60% suspended solids. A portion of the slurry of this example, prior to separation of the suspended solids from the liquid, provided a sample for the analyses shown in Table 3. The aspect ratio of the microcrystalline cellulose particles of this example was in the range of 4.00 to 6.5. The lignin content of this example was determined to be 13.6%.

### Example 6

A slurry of 10.0% concentration of oat hulls in water, and sufficient sulfuric acid to provide a pH of 1.9 were mixed with water heated to a sub-critical temperature. The combination of both streams produced a reactor inlet temperature targeted at 235° C. The reactor pressure was controlled by a let-down valve at a pressure of 2440 psi. The reaction time (time that a particle of the cellulose-containing feedstock spent in the reactor, on average) was 1.2 seconds. The product from this example yielded 51% suspended solids. A portion of the slurry of this example, prior to separation of the suspended solids from the liquid, provided a sample for the analyses shown in Table 3. The aspect ratio of the microcrystalline cellulose particles of this example was in the range of 3.53 to 5.03. The weight average molecular weight of the cellulose prepared in this example was determined to be 28,508 grams per mole. The lignin content of this example was determined to be 49.3%.

### Example 7

### Skin Moisturizer

A mixture was prepared by combining the suspended solids from Example 1 (having a moisture content of 84.9%) (10 wt %), dehydroxanthan gum (0.2 wt %), ascorbic acid (5.0%), and deionized water, qsp to a total of 100%. This mixture was poured into a mold and dried at 50° C. for 18 hours to give a thin film. 0.05 g of this film was placed on the back of the hands of volunteers, rehydrated with 1 ml water, and rubbed until fully absorbed on the skin. The moisture and oil content on the skin was evaluated at baseline, 10 minutes after application, and 60 minutes after application.

### Example 8

### Skin Moisturizer

A mixture was prepared by combining a concentrated portion of the suspended solids and dissolved solids from Example 1, having a moisture content of 84.9%, (10 wt %), dehydroxanthan gum (0.2 wt %), ascorbic acid (5.0%), and deioinized water, qsp to a total of 100%. This mixture was poured into a mold and dried at 50° C. for 18 hours to give a thin film. 0.05 g of this film was placed on the back of the hands of volunteers, rehydrated with 1 ml water, and rubbed until fully absorbed on the skin. The moisture and oil content on the skin was evaluated at baseline, 10 minutes after application, and 60 minutes after application.

For both Examples 7 and 8, a Digital Moisture Oil Content Analyzer SK-8 (FATUXZ) was used to observe changes in skin moisture and oil content. The skin on the back of the hands normally presents a moisture content of between 30% and 60%, while an oil content between 8% and 18% is characteristic of dry skin, and an oil content between 18% and 30% is characteristic of normal skin.

Table 4 shows the results of these Examples

TABLE 4

| | Moisture Content (%) | | Oil Content (%) | |
|---|---|---|---|---|
| | Example 7 | Example 8 | Example 7 | Example 8 |
| Baseline | 32.4 | 33.3 | 14.6 | 14.9 |
| After 10 min | 33.9 | 34.7 | 15.2 | 15.6 |
| After 60 min | 35.6 | 36.5 | 16.0 | 16.4 |
| % Increase at 60 min | 3.2 | 1.4 | 3.2 | 1.5 |

These data show that the formulations of both examples positively affected the skin's moisture and oil content, providing increased hydration over time. After application, volunteers found that their skin also felt more supple and smooth, based on qualitative sensorial evaluation.

### Example 9

### Egg-Free Mayonnaise

6.0 g of an aqueous slurry of a concentrated product from Example 1, that contained 15.1% of the suspended solids of Example 1, and 1.4% of the dissolved solids of Example 1, the remainder being water, was combined with 1.20 g of yellow mustard, 2.39 g of white distilled vinegar, 0.13 g guar, and 0.04 g of salt. This mixture was mixed at 2,500 rpm with a propeller mixer. Sunflower oil was added dropwise to the mixing slurry. After approximately 31 g of sunflower oil had been added, the emulsion was thick and stable and resembled mayonnaise.

### Example 10

### Egg-Free Mayonnaise

A similar mayonnaise was generated in a substantially similar procedure to that of Example 9, using an aqueous slurry that contained 10.0% of suspended solids, the remainder being water, from Example 1.

It will be apparent to those skilled in the art that various modifications and variations can be made in the present invention without departing from the scope or spirit of the invention. Other aspects of the invention will be apparent to those skilled in the art from consideration of the specification and practice of the invention disclosed herein. It is intended that the specification and examples be considered as exemplary only, with a true scope and spirit of the invention being indicated by the following claims.

1. A method for the preparation of a cellulose-containing composition comprising the steps of:
(a) forming a first slurry comprising a cellulose-containing feedstock, water, and acid;
(b) subjecting the first slurry to rapid heating to a first temperature, by addition of subcritical, near critical or supercritical water, at a first pressure, thereby forming a second slurry;
(c) maintaining the second slurry at the first temperature and first pressure for a first time period;
(d) rapidly depressurizing the second slurry to form a third slurry at a second temperature and a second pressure; and
(e) separating the liquid portion from the solid portion of the third slurry, the solid portion of which comprises the cellulose-containing composition;
wherein the first temperature is in the range of 180° C. to 280° C.; and
wherein the cellulose contained in the cellulose-containing composition has a weight average molecular weight between 40,000 grams/mole and 155,000 grams/mole.

2. The method according to claim 1, wherein the cellulose contained in the cellulose-containing composition has a weight average molecular weight between 75,000 grams/mole and 155,000 grams/mole.

3. The method according to claim 1, wherein the cellulose-containing feedstock comprises cellulose, hemicellulose, or lignin, or a mixture thereof.

4. The method according to claim 1, wherein the cellulose-containing feedstock is selected from woody biomass, pea hulls, corn fiber, soy hulls, citrus residues, cocoa shells, cotton, oat fiber, bran fiber, bagasse, crop grasses, hemp, cereals, fruits, vegetables, paper products and wood products, and mixtures of two or more thereof.

5. The method according to claim 1, wherein the first temperature is in the range from 200° C. to less than or equal to 260° C.

6. The method according to claim 1, wherein the first time period is from 0.5 second to 4 seconds.

7. The method according to claim 1, wherein the rapidly depressurizing occurs over a period of less than 5% of the first time period.

8. The method according to claim 1, wherein the rapid heating occurs over a period of from 5% to 50% of the first time period.

9. The method according to claim 1, wherein the first pressure is in the range from 1,500 psi to 3,600 psi.

10. The method according to claim 1, wherein the second pressure is in the range of 5 psi to 75 psi.

11. The method according to claim 1, wherein the second temperature is in the range from 45° C. to 150° C.

12. (canceled)

13. The method according to claim 1, wherein the acid is an inorganic acid selected from sulfuric acid, sulfonic acid, phosphoric acid, phosphonic acid, nitric acid, nitrous acid, hydrochloric acid, hydrofluoric acid, hydrobromic acid, and hydroiodic acid, and mixtures thereof.

14. (canceled)

15. The method according to claim 1, wherein the acid is an organic acid selected from an aliphatic carboxylic acid, an aromatic carboxylic acid, a dicarboxylic acid, an aliphatic fatty acid, an aromatic fatty acid, an amino acid, an alkyl sulfonic acid, and an acyl sulfonic acid, and mixtures thereof.

16-21. (canceled)

22. The method according to claim 1, wherein the pH of the first slurry is in the range from pH 1.0 to pH 3.0.

23. (canceled)

24. The method according to claim 1, wherein the cellulose-containing composition comprises from 60% to 90% cellulose based on the total weight of the cellulose-containing composition.

25. The method according to claim 1, wherein the cellulose-containing composition comprises microcrystalline cellulose.

26. The method according to claim 1, wherein the cellulose-containing composition comprises at least 50% microcrystalline cellulose based on the total weight of the cellulose-containing composition.

27-30. (canceled)

31. The method according to claim 1, wherein the cellulose-containing composition further comprises lignin or hemicellulose, or a mixture thereof.

32-33. (canceled)

34. The method according to claim 1, wherein the particles of the cellulose-containing composition have a mean particle size between 20 μm and 110 μm.

35. (canceled)

36. A cellulose-containing composition prepared by the method according to claim 1.

37-123. (canceled)
